(19) United States
(12) Patent Application Publication
(10) Pub. No.: US 2026/0157721 A1
(43) Pub. Date: Jun. 11, 2026

(54) SYSTEMS AND METHODS FOR DETERMINING EVALUATION PARAMETERS OF HEARTS

(71) Applicant: WUHAN UNITED IMAGING HEALTHCARE CO., LTD., Wuhan (CN)

(72) Inventor: Rui BIAN, Wuhan (CN)

(73) Assignee: WUHAN UNITED IMAGING HEALTHCARE CO., LTD., Wuhan (CN)

(21) Appl. No.: 19/411,418

(22) Filed: Dec. 8, 2025

(30) Foreign Application Priority Data
Dec. 6, 2024 (CN) 202411796751.9

(51) Int. Cl. A61B 8/06; A61B 8/00

(52) U.S. Cl. CPC A61B 8/06; A61B 8/488; A61B 8/5223

(57) ABSTRACT

The present disclosure provides a system and method for determining one or more evaluation parameters of a heart. The method is performed by a computing device including at least one processor and at least one storage device. The method includes obtaining a Doppler ultrasound spectral image of the heart; determining a plurality of cardiac cycles based on the Doppler ultrasound spectral image; and determining the one or more evaluation parameters for evaluating a function of the heart based on the plurality of cardiac cycles and the Doppler ultrasound spectral image.

### CROSS-REFERENCE TO RELATED APPLICATIONS

This application claims the priority of Chinese Patent Application No. 202411796751.9, filed on December 6, 2024, the contents of which are hereby incorporated by reference.

### TECHNICAL FIELD

The disclosure generally relates to the field of medical technology, and in particular relates to systems and methods for determining evaluation parameters of hearts.

### BACKGROUND

In conventional cardiac ultrasound examinations, doctors can, as needed, use tissue Doppler imaging (TDI) in the apical four-chamber view to obtain motion velocity spectrograms of the patient's mitral annular septal wall, mitral annular lateral wall, tricuspid annular lateral wall, etc. From these spectrograms, doctors can directly obtain the evaluation parameters of hearts through manual measurement: early diastolic motion velocity (e'), late diastolic motion velocity (a'), systolic motion velocity (S'), isovolumic relaxation time (IVRT), isovolumic contraction time (IVCT), and ejection time (ET). In actual examinations, this method, which relies entirely on manual measurement by doctors, requires doctors to repeatedly use measuring tools to measure points and time periods, which leads to excessively long and inefficient cardiac ultrasound examinations. Meanwhile, since the boundaries of IVCT, IVRT, and ET are not clear enough and their intervals are small, large deviations are prone to occur when clinical doctors lack sufficient experience or operate carelessly, resulting in increased measurement errors. Thus, it is desirable to provide a system and method for determining evaluation parameters of hearts with higher accuracy.

### SUMMARY

According to a first aspect of the present disclosure, a method for determining one or more evaluation parameters of a heart is provided. The method is performed by a computing device including at least one processor and at least one storage device. The method includes: obtaining a Doppler ultrasound spectral image of the heart; determining a plurality of cardiac cycles based on the Doppler ultrasound spectral image; and determining the one or more evaluation parameters for evaluating a function of the heart based on the plurality of cardiac cycles and the Doppler ultrasound spectral image.

In some embodiments, the determining a plurality of cardiac cycles based on the Doppler ultrasound spectral image includes: dividing, based on a horizontal axis in the Doppler ultrasound spectral image, the Doppler ultrasound spectral image into a first Doppler ultrasound spectral image and a second Doppler ultrasound spectral image, the first Doppler ultrasound spectral image being a portion of the Doppler ultrasound spectral image above the horizontal axis, the second Doppler ultrasound spectral image being a portion of the Doppler ultrasound spectral image below the horizontal axis; and determining the plurality of cardiac cycles based on the first Doppler ultrasound spectral image and the second Doppler ultrasound spectral image.

In some embodiments, the one or more evaluation parameters include cardiac function evaluation parameters related to systole and cardiac function evaluation parameters related to diastole. The determining the one or more evaluation parameters for evaluating a function of the heart based on the plurality of cardiac cycles and the Doppler ultrasound spectral image includes:determining the cardiac function evaluation parameters related to the systole based on the plurality of cardiac cycles and the first Doppler ultrasound spectral image; and determining the cardiac function evaluation parameters related to the diastole based on the cardiac function evaluation parameters related to the systole and the second Doppler ultrasound spectral image.

In some embodiments, the determining the one or more evaluation parameters for evaluating a function of the heart based on the plurality of cardiac cycles and the Doppler ultrasound spectral image includes: determining first target key point positions of the systole in each cardiac cycle of the plurality of cardiac cycles of the first Doppler ultrasound spectral image using a key point detection model, wherein the key point detection model is a machine learning model, and the first target key point positions are points for extracting the cardiac function evaluation parameters related to the systole; determining second target key point positions in the diastole in each cardiac cycle of the plurality of cardiac cycles of the second Doppler ultrasound spectral image based on the cardiac function evaluation parameters related to the systole using the key point detection model, wherein the second target key point positions are points for extracting cardiac function evaluation parameters related to the diastole; and determining the one or more evaluation parameters based on the first target key point positions and the second target key point positions.

In some embodiments, the determining a plurality of cardiac cycles based on the Doppler ultrasound spectral image includes: performing, based on a horizontal axis in the Doppler ultrasound spectral image, a segmentation on the Doppler ultrasound spectral image to obtain a first Doppler ultrasound spectral image above the horizontal axis; and determining the plurality of cardiac cycles based on the first Doppler ultrasound spectral image.

In some embodiments, the determining the plurality of cardiac cycles based on the first Doppler ultrasound spectral image includes: generating an intermediate curve based on the first Doppler ultrasound spectral image; determining a plurality of minimum value positions in the intermediate curve; and determining the plurality of cardiac cycles based on the plurality of minimum value positions.

In some embodiments, the determining a plurality of minimum value positions in an intermediate curve generated based on the first Doppler ultrasound spectral image includes: binarizing the first Doppler ultrasound spectral image; summing pixel values on a vertical axis of the binarized first Doppler ultrasound spectral image and performing smoothing processing to obtain the intermediate curve; and determining the plurality of minimum value positions based on the intermediate curve.

In some embodiments, the determining the one or more evaluation parameters for evaluating a function of the heart based on the plurality of cardiac cycles and the Doppler ultrasound spectral image includes: dividing the first Doppler ultrasound spectral image into a plurality of first sub-images based on the plurality of cardiac cycles; for each first sub-image of the plurality of first sub-images, determining a first key point heatmap set of the first sub-image and a first score of the first sub-image by inputting the first sub-image into a key point detection model, wherein the key point detection model is a machine learning model, and the first score reflects a recognition quality of a target key point in the first sub-image, the target key point is a point for extracting cardiac function evaluation parameters related to a cardiac cycle; determining one or more first target sub-images from the plurality of first sub-images based on first scores of the plurality of first sub-images; and determining first target key point positions of systole in a cardiac cycle corresponding to each first target sub-image of the one or more first target sub-images by post-processing the first key point heatmap set of the first target sub-image, the first target key point positions are points for extracting cardiac function evaluation parameters related to the systole.

In some embodiments, the first target key point positions of the systole includes a peak position of systolic motion velocity, a start position of an ejection phase, and an end position of the ejection phase. The determining the one or more evaluation parameters for evaluating a function of the heart based on the plurality of cardiac cycles and the Doppler ultrasound spectral image further includes: mapping peak positions of systolic motion velocity, start positions of the ejection phase, and end positions of the ejection phase in a plurality of first cardiac cycles to the Doppler ultrasound spectral image, wherein each of the plurality of first cardiac cycles is a cardiac cycle corresponding to each of the one or more first target sub-images; determining a plurality of second sub-images by performing a period division on a second Doppler ultrasound spectral image that is below the horizontal axis in the Doppler ultrasound spectral image based on the peak positions of systolic motion velocity corresponding to the plurality of first cardiac cycles; for each second sub-image of the plurality of second sub-images, determining a second key point heatmap set of the second sub-image and a second score of the second sub-image by inputting the second sub-image into the key point detection model, the second score reflects a recognition quality of the target key point in the second sub-image; determining one or more second target sub-images from the plurality of second sub-images based on second scores of the plurality of second sub-images; and determining second target key point positions in diastole in a cardiac cycle corresponding to each second target sub-image of the one or more second target sub-images by post-processing the second key point heatmap set of the second target sub-image, the second target key point positions are points for extracting cardiac function evaluation parameters related to the diastole.

In some embodiments, the second target key point positions in the diastole includes a peak position and a start position of early diastolic motion velocity, and a peak position and an end position of late diastolic motion velocity. The determining the one or more evaluation parameters for evaluating a function of the heart based on the plurality of cardiac cycles and the Doppler ultrasound spectral image further includes: determining a target Doppler ultrasound spectral image by marking the peak positions of systolic motion velocity, the start positions and the end positions of the ejection phase, peak positions and start positions of early diastolic motion velocity, and peak positions and end positions of late diastolic motion velocity in a plurality of second cardiac cycles on the Doppler ultrasound spectral image, wherein each of the plurality of second cardiac cycles is a cardiac cycle corresponding to each of the one or more second target sub-images; and determining the one or more evaluation parameters based on the target Doppler ultrasound spectral image.

In some embodiments, the one or more evaluation parameters includes at least one of a peak systolic tissue velocity S', a peak early diastolic tissue velocity e', a peak late diastolic tissue velocity a', an isovolumic contraction time (IVCT), an ejection time (ET), and an isovolumic relaxation time (IVRT). Determining the one or more evaluation parameters based on the target Doppler ultrasound spectral image includes at least one of: determining a velocity corresponding to a peak position of systolic motion velocity in the target Doppler ultrasound spectral image as the peak systolic tissue velocity S'; determining a velocity corresponding to a peak position of early diastolic motion velocity in the target Doppler ultrasound spectral image as the peak early diastolic tissue velocity e'; determining a velocity corresponding to a peak position of late diastolic motion velocity in the target Doppler ultrasound spectral image as the peak late diastolic tissue velocity a'; determining a time from an end position of late diastolic motion velocity to a start position of an ejection phase adjacent on a right side in the target Doppler ultrasound spectral image as the IVCT; determining a time between the start position and the end position of the ejection phase in the target Doppler ultrasound spectral image as the ejection time; or determining a time from the end position of the ejection phase to a start position of early diastolic motion velocity of an early diastole adjacent on a right side in the target Doppler ultrasound spectral image as the IVRT.

In some embodiments, the method further includes: determining a target peak systolic tissue velocity S'’ by calculating an average value of peak systolic tissue velocities S' in the plurality of cardiac cycles, or by determining a peak systolic tissue velocity S' in one cardiac cycle based on a first selection instruction; determining a target peak early diastolic tissue velocity e'’ by calculating an average value of peak early diastolic tissue velocities e' in the plurality of cardiac cycles, or by determining a peak early diastolic tissue velocity e' in one cardiac cycle based on a second selection instruction; determining a target peak late diastolic tissue velocity a'’ by calculating an average value of peak late diastolic tissue velocities a' in the plurality of cardiac cycles, or by determining a peak late diastolic tissue velocity a' in one cardiac cycle based on a third selection instruction; determining a target IVCT by calculating an average value of IVCTs in the plurality of cardiac cycles, or by determining an IVCT in one cardiac cycle based on a fourth selection instruction; determining a target ejection time by calculating an average value of ejection times in the plurality of cardiac cycles, or by determining an ejection time in one cardiac cycle based on a fifth selection instruction; and determining a target IVRT by calculating an average value of IVRTs in the plurality of cardiac cycles, or by determining an IVRT in one cardiac cycle based on a sixth selection instruction.

In some embodiments, the method further includes: calculating a standard deviation of target parameters in the plurality of second cardiac cycles, wherein the target parameters are at least one of the one or more evaluation parameters; and determining a plurality of target cardiac cycles from the plurality of second cardiac cycles based on the standard deviation of the target parameters.

In some embodiments, training samples of the key point detection model includes a first training sample set, a second training sample set, a third training sample set, and a fourth training sample set. The first training sample set includes a plurality of sample first sub-images, the second training sample set includes a plurality of sample second sub-images, the third training sample set includes a plurality of complete sample first sub-images and a plurality of incomplete sample first sub-images, and the fourth training sample set includes a plurality of complete sample second sub-images and a plurality of incomplete sample second sub-images. A training process of the key point detection model includes: performing a plurality of iterations on an initial key point detection model based on a target loss function using the first training sample set, the second training sample set, the third training sample set, and the fourth training sample set until a convergence condition is reached. The target loss function includes a first loss term, a second loss term, a third loss term, and a fourth loss term. The first loss term reflects a positioning accuracy of a target key point position in the systole. The second loss term reflects a positioning accuracy of a target key point position in a diastole. The third loss term reflects an accuracy of quality assessment of each sample first sub-image. The fourth loss term reflects an accuracy of quality assessment of each sample second sub-image.

In some embodiments, the target loss function further includes a fifth loss term, the fifth loss term reflecting a local consistency and a unimodality of a predicted key point.

In some embodiments, the training process of the key point detection model further includes: determining a convergence speed of each loss term during one or more preceding rounds of training; determining a weight corresponding to each loss term based on the convergence speed of each loss term; and determining a value of the target loss function by weighting and summing all loss terms of the target loss function based on weights of the all loss terms.

According to a second aspect of the present disclosure, a system is provided. The system may include at least one storage device storing executable instructions for determining one or more evaluation parameters of a heart, and at least one processor in communication with the at least one storage device. When executing the executable instructions, the at least one processor may cause the system to perform one or more of the following operations. The system may obtain a Doppler ultrasound spectral image of the heart; determine a plurality of cardiac cycles based on the Doppler ultrasound spectral image; and determine the one or more evaluation parameters for evaluating a function of the heart based on the plurality of cardiac cycles and the Doppler ultrasound spectral image.

In some embodiments, to determine the plurality of cardiac cycles based on the Doppler ultrasound spectral image, the system may divide, based on a horizontal axis in the Doppler ultrasound spectral image, the Doppler ultrasound spectral image into a first Doppler ultrasound spectral image and a second Doppler ultrasound spectral image. The first Doppler ultrasound spectral image is a portion of the Doppler ultrasound spectral image above the horizontal axis. The second Doppler ultrasound spectral image is a portion of the Doppler ultrasound spectral image below the horizontal axis. The system may determine the plurality of cardiac cycles based on the first Doppler ultrasound spectral image and the second Doppler ultrasound spectral image.

In some embodiments, the one or more evaluation parameters including cardiac function evaluation parameters related to systole and cardiac function evaluation parameters related to diastole. To determine the one or more evaluation parameters for evaluating a function of the heart based on the plurality of cardiac cycles and the Doppler ultrasound spectral image, the system may determine the cardiac function evaluation parameters related to the systole based on the plurality of cardiac cycles and the first Doppler ultrasound spectral image; and determine the cardiac function evaluation parameters related to the diastole based on the cardiac function evaluation parameters related to the systole and the second Doppler ultrasound spectral image.

According to a third aspect of the present disclosure, a non-transitory computer-readable medium storing at least one set of instructions for determining one or more evaluation parameters of a heart is provided. When executed by at least one processor, the at least one set of instructions may direct the at least one processor to perform a method. The method may include obtaining a Doppler ultrasound spectral image of the heart; determining a plurality of cardiac cycles based on the Doppler ultrasound spectral image; and determining the one or more evaluation parameters for evaluating a function of the heart based on the plurality of cardiac cycles and the Doppler ultrasound spectral image.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not scaled. These embodiments are non-limiting exemplary embodiments, in which like reference numerals represent similar structures throughout the several views of the drawings, and wherein:

FIG. 1 is a schematic diagram illustrating an exemplary ultrasound imaging system according to some embodiments of the present disclosure;

FIG. 2A is a block diagram illustrating an exemplary processing device according to some embodiments of the present disclosure;

FIG. 2B is a block diagram illustrating an exemplary processing device according to some other embodiments of the present disclosure;

FIG. 3A is a schematic flowchart illustrating an exemplary process for determining one or more evaluation parameters of a heart according to some embodiments of the present disclosure;

FIG. 3B is a schematic flowchart illustrating an exemplary process for determining one or more evaluation parameters of a heart according to some embodiments of the present disclosure;

FIG. 4 is a schematic diagram illustrating determining target key point positions and scores of sub-images using an exemplary key point detection model according to some embodiments of the present disclosure;

FIG. 5 shows a measurement result according to some embodiments of the present disclosure;

FIG. 6 is a schematic flowchart illustrating an exemplary process for determining one or more evaluation parameters of a heart according to some embodiments of the present disclosure;

FIG. 7A is a schematic flowchart illustrating an exemplary process for determining a plurality of first sub-images according to some embodiments of the present disclosure;

FIG. 7B is a schematic diagram illustrating a first sub-image marked with first target key points according to some embodiments of the present disclosure;

FIG. 8A is a schematic flowchart illustrating an exemplary process for determining a plurality of second sub-images according to some embodiments of the present disclosure;

FIG. 8B is a schematic diagram illustrating a second sub-image marked with second target key points according to some embodiments of the present disclosure; and

FIG. 9 is a schematic flowchart illustrating an exemplary training process of a key point detection model according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, it should be understood that terms such as "center,” “longitudinal,” “transverse,” “length,” “width,” “thickness,” “upper,” “lower,” ‘front,” “rear,” “left,” “right,” “vertical,” “horizontal,” “top,” “bottom,” “inner,” “outer,” “clockwise,” “counterclockwise,” “axial,” “radial,” “circumferential,” etc., are used to indicate orientations or positional relationships based on those shown in the accompanying drawings. These terms are used solely to facilitate the description of this application and to simplify the description, and do not indicate or imply that the referred devices or elements must have a specific orientation or be constructed and operated in a specific orientation. Therefore, they should not be construed as limitations on this application.

The flowcharts used in the present disclosure illustrate operations that systems implement according to some embodiments in the present disclosure. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

The term “image” in the present disclosure is used to collectively refer to imaging data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “region,” “location,” and "area" in the present disclosure may refer to a location of an anatomical structure shown in the image or an actual location of the anatomical structure existing in or on a target object’s body, since the image may indicate the actual location of a certain anatomical structure existing in or on the target object’s body.

Provided herein are a system and method for determining one or more evaluation parameters of a heart. The method may be performed by a computing device including at least one processor and at least one storage device. The method may include acquiring a Doppler ultrasound spectral image of the heart and determining a plurality of cardiac cycles based on the Doppler ultrasound spectral image. The method may further include determining the one or more evaluation parameters for evaluating a function of the heart based on the plurality of cardiac cycles and the Doppler ultrasound spectral image.

According to some embodiments of the present disclosure, compared with traditional cardiac examination techniques, by determining the plurality of cardiac cycles based on the Doppler ultrasound spectral image, and further determining the evaluation parameters based on the plurality of cardiac cycles and the Doppler ultrasound spectral image, the evaluation parameters can be automatically recognized without relying on manual operation by doctors using measuring tools, thereby improving the detection efficiency and enhancing the accuracy of the determined evaluation parameters. Additionally, unlike manual measurement, which usually only provides measurement results for one cardiac cycle, the proposed method can generate measurement results for all cardiac cycles in the Doppler ultrasound spectral image, making the final measured values more reasonable.

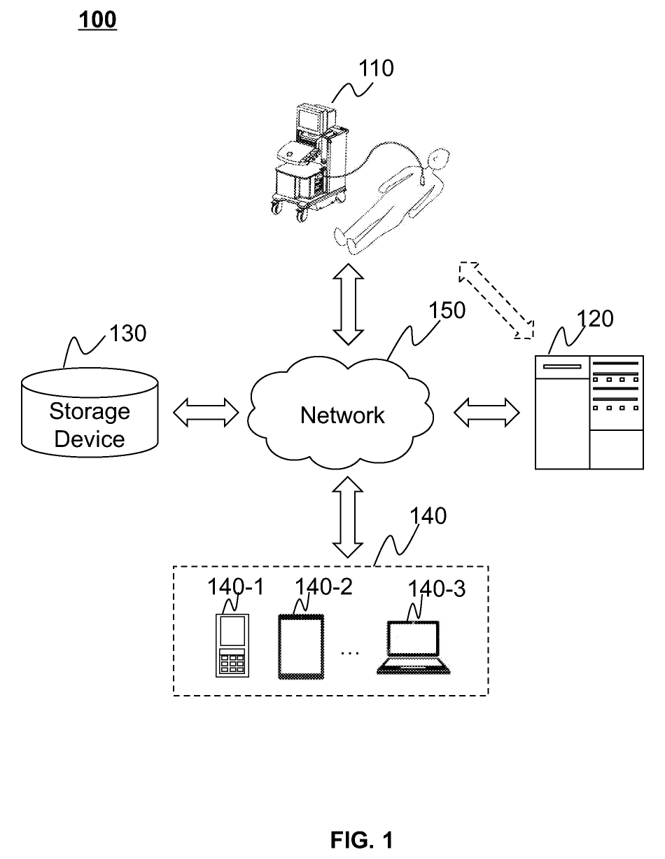

FIG. 1 is a schematic diagram illustrating an exemplary ultrasound imaging system 100 according to some embodiments of the present disclosure. As shown in FIG. 1, the ultrasound imaging system 100 may include a medical device 110, a processing device 120, a storage device 130, a terminal device 140, and a network 150. The components in the ultrasound imaging system 100 may be connected to and/or communicate with each other via a wireless connection, a wired connection, or a combination thereof.

The medical device 110 may be configured to acquire ultrasound imaging data relating to at least one part of a subject to generate an image of the at least one part of the subject. In some embodiments, the ultrasound imaging data may be a two-dimensional (2D) imaging data, a three-dimensional (3D) imaging data, a four- dimensional (4D) imaging data, or the like, or any combination thereof. The subject may be biological or non-biological. For example, the subject may include a patient, a man-made object, etc. As another example, the subject may include a specific portion, organ, and/or tissue of the patient. For example, the subject may include the heart, thyroid gland, esophagus, trachea, stomach, gallbladder, small intestine, colon, bladder, uterus, fallopian tubes, or the like, or any combination thereof.

In some embodiments, the medical device 110 may be a medical imaging device based on an ultrasound mode, for example, a Doppler ultrasound diagnosis device, an ultrasound diagnosis instrument, an ultrasound Doppler flow analyzer, etc. The above medical device 110 is merely provided for the purposes of illustration and is not a limitation on the scope. The medical device 110 may obtain a medical ultrasound image associated with a blood flow velocity based on a Doppler effect. In some embodiments, the medical device 110 may obtain the medical ultrasound image of a patient, e.g., a spectral Doppler blood flow image, etc., and send the medical ultrasound image to the processing device 120. In some embodiments, the medical device 110 may exchange data and/or information with other components (e.g., the processing device 120, the storage device 130, the terminal device 140) of the ultrasound imaging system 100 through the network 150. In some embodiments, one or more components (e.g., the processing device 120, the storage device 130, the terminal device 140) of the system 100 may be included in the medical device 110.

The processing device 120 may process data and/or information relating to evaluation parameter determination to perform one or more functions described in the present disclosure. For example, the processing device 120 may acquire a Doppler ultrasound spectral image of the heart of a patient. The processing device 120 may determine a plurality of cardiac cycles based on the Doppler ultrasound spectral image. Further, the processing device 120 may determine one or more evaluation parameters for evaluating a function of the heart based on the plurality of cardiac cycles and the Doppler ultrasound spectral image. In some embodiments, the processing device 120 may be a computer, a user console, a single server, or a server group, etc. The server group may be centralized or distributed. In some embodiments, the processing device 120 may be local or remote. In some embodiments, the processing device 120 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing device 120 may be implemented on a computing device or the medical device 110.

The storage device 130 may be configured to store data and/or instructions. The data and/or instructions may be obtained from, for example, the processing device 120, the medical device 110, and/or any other component of the ultrasound imaging system 100. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or any combination thereof. In some embodiments, the storage device 130 may be implemented on the cloud platform described elsewhere in the present disclosure.

The terminal device 140 may be configured to receive information and/or data from the processing device 120, the medical device 110, and/or the storage device 130 via the network 150. For example, the terminal device 140 may receive an image from the processing device 120. In some embodiments, the terminal device 140 may provide a user interface via which a user may view information and/or input data and/or instructions to the ultrasound imaging system 100. For example, the user may view, via the user interface, information associated with the medical device 110. As another example, the user may input, via the user interface, a user input instruction to adjust imaging parameters for re-imaging. In some embodiments, the terminal device 140 may include a mobile device 140-1, a tablet computer 140-2, a laptop computer 140-3, or the like, or any combination thereof. In some embodiments, the terminal device 140 may include a display that can display information in a human-readable form, such as text, image, audio, video, graph, animation, or the like, or any combination thereof.

The network 150 may facilitate the exchange of information and/or data for the ultrasound imaging system 100. In some embodiments, one or more components (e.g., the medical device 110, the processing device 120, the terminal device 140, or the storage device 130) of the ultrasound imaging system 100 may transmit information and/or data to one or more other components of the ultrasound imaging system 100 via the network 150. In some embodiments, the network 150 may be any type of wired or wireless network, or combination thereof.

It should be noted that the above description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. In some embodiments, the ultrasound imaging system 100 may include one or more additional components and/or one or more components described above may be omitted. Additionally or alternatively, two or more components of the ultrasound imaging system 100 may be integrated into a single component. For example, the processing device 120 may be integrated into the medical device 110. As another example, a component of the ultrasound imaging system 100 may be replaced by another component that can implement the functions of the component. However, those variations and modifications do not depart from the scope of the present disclosure.

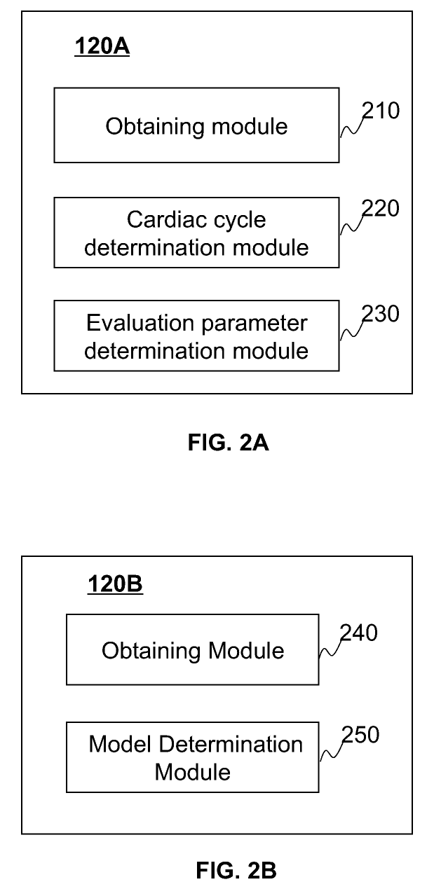

FIG. 2A is a block diagram illustrating an exemplary processing device 120A according to some embodiments of the present disclosure. FIG. 2B is a block diagram illustrating an exemplary processing device 120B according to some embodiments of the present disclosure. The processing devices 120A and 120B may be exemplary processing devices 120 as described in connection with FIG. 1. In some embodiments, the processing devices 120A and 120B may be respectively implemented on a processing unit. Alternatively, the processing devices 120A and 120B may be implemented on a same computing unit.

As illustrated in FIG. 2A, the processing device 120A may include an obtaining module 210, a cardiac cycle determination module 220, and an evaluation parameter determination module 230.

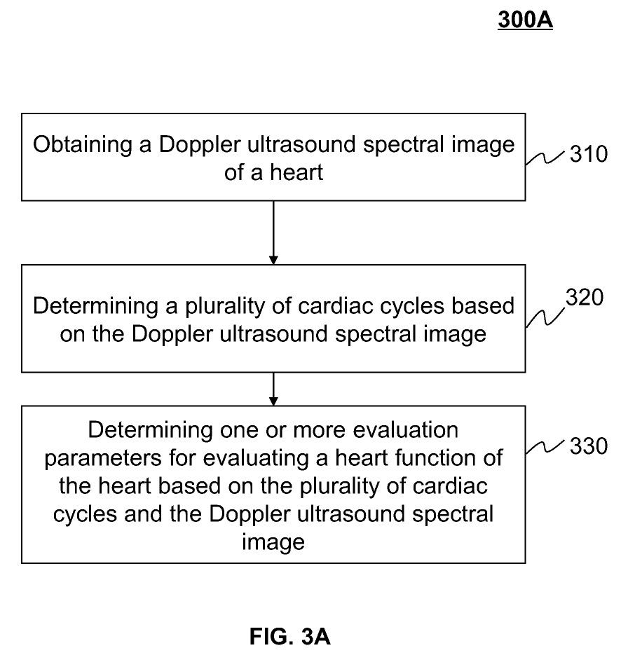

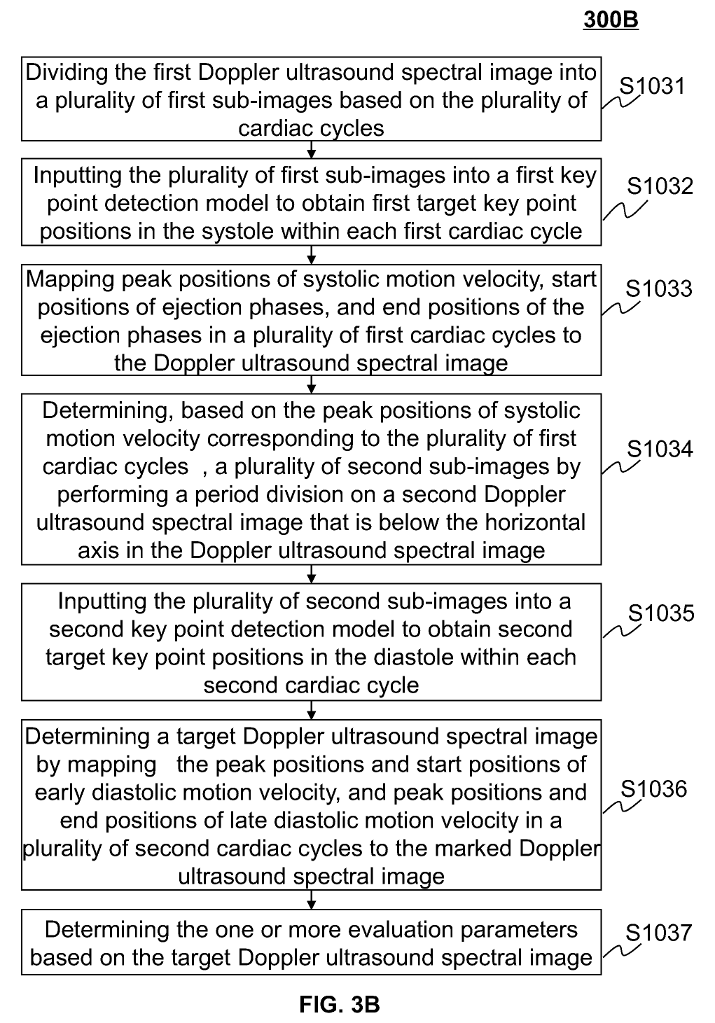

The acquisition module 210 may be configured to obtain a Doppler ultrasound spectral image of the heart. More descriptions regarding the obtaining of the Doppler ultrasound spectral image may be found elsewhere in the present disclosure, e.g., operation 310 in FIG. 3 and relevant descriptions thereof.

The cardiac cycle determination module 220 may be configured to determine a plurality of cardiac cycles based on the Doppler ultrasound spectral image. More descriptions regarding the determination of the cardiac cycles may be found elsewhere in the present disclosure, e.g., operation 320 in FIG. 3 and relevant descriptions thereof.

The evaluation parameter determination module 230 may be configured to determine the one or more evaluation parameters for evaluating a function of the heart based on the plurality of cardiac cycles and the Doppler ultrasound spectral image. More descriptions regarding the determination of the evaluation parameters may be found elsewhere in the present disclosure, e.g., operation 330 in FIG. 3 and relevant descriptions thereof.

As illustrated in FIG. 2B, the processing device 120B may include an obtaining module 240 and a model determination module 250.

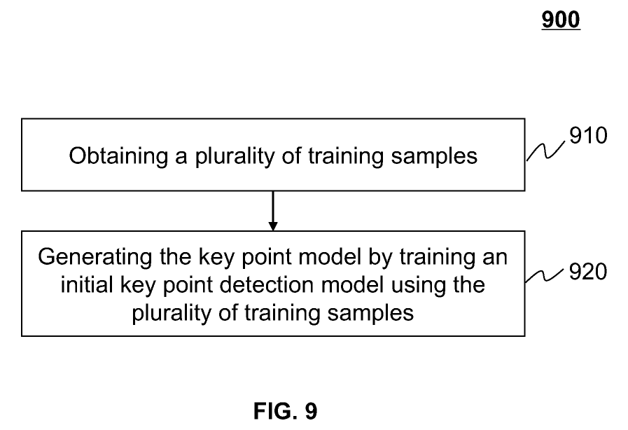

The obtaining module 240 may be configured to a plurality of training samples. More descriptions regarding the obtaining of the training samples may be found elsewhere in the present disclosure, e.g., operation 910 in FIG. 9 and relevant descriptions thereof.

The model determination module 250 may be configured to generate a key point detection model by training an initial key point detection model using the plurality of training samples. More descriptions regarding the training of the key point detection model may be found elsewhere in the present disclosure, e.g., operation 920 in FIG. 9 and relevant descriptions thereof.

Each of the modules described above may be a hardware circuit that is designed to perform certain actions, e.g., according to a set of instructions stored in one or more storage media, and/or any combination of the hardware circuit and the one or more storage media.

It should be noted that the above description is merely provided for the purposes of illustration and is not intended to limit the scope of the present disclosure. Apparently, for persons having ordinary skills in the art, multiple variations and modifications may be conducted under the teachings of the present disclosure. However, those variations and modifications do not depart from the scope of the present disclosure. In some embodiments, the processing device 120A and/or the processing device 120B may share two or more of the modules, and any one of the modules may be divided into two or more units. For instance, the processing devices 120A and 120B may share a same obtaining module; that is, the obtaining module 210 and the obtaining module 240 are a same module. In some embodiments, the processing device 120A and/or the processing device 120B may include one or more additional modules, such as a storage module (not shown) for storing data. In some embodiments, the processing device 120A and the processing device 120B may be integrated into one processing device 120.

FIG. 3A is a schematic flowchart illustrating an exemplary process 300A for determining one or more evaluation parameters of a heart according to some embodiments of the present disclosure. In some embodiments, the process 300A may be automatically executed by the ultrasound imaging system 100. For example, the process 300A may be implemented as a set of instructions (e.g., an application) stored in the storage device 130, and the processing device 120 (e.g., the modules in FIG. 2A) may execute the set of instructions and may accordingly be directed to perform the process 300A. The operations of the illustrated process presented below are intended to be illustrative. In some embodiments, the process 300A may be accomplished with one or more additional operations not described and/or without one or more of the operations discussed. Additionally, the order of the operations of the process 300A illustrated in FIG. 3A and described below is not intended to be limiting.

In some embodiments, the process 300A may be applied to an electronic device such as a mobile phone, a tablet computer, a wearable device, an in-vehicle device, an augmented reality (AR)/virtual reality (VR) devics, a laptop, an ultra-mobile personal computer (UMPC), a netbook, a personal digital assistant (PDA), an electrosurgical host, etc. The embodiments of the present disclosure do not impose any limitation on the specific type of electronic device.

In 310, the processing device 120 (e.g., the obtaining module 210) may obtain a Doppler ultrasound spectral image of the heart.

The Doppler ultrasound spectral image refers to an image obtained through techniques such as echocardiography. In an echocardiographic examination, the Doppler effect may be utilized to measure velocity information such as blood flow velocity and myocardial motion velocity in the heart, and these velocity information may be displayed in a form of a spectral image (i.e., the Doppler ultrasound spectral image). The abscissa (also referred to as a horizontal axis or time axis) of the Doppler ultrasound spectral image represents sampling time. Each point on the horizontal axis represents a time point. The ordinate (also referred to as a vertical axis or velocity axis) represents the motion velocity (such as blood flow velocity or myocardial motion velocity) associated with the heart. Each point on the vertical axis represents a velocity value.

In some embodiments, the Doppler ultrasound spectral image may be obtained from a medical device (e.g., the medical device 110), the storage device 130, or any other storage device. For example, an ultrasound probe of the medical device (or the ultrasound imaging system 100) may emit ultrasonic waves to the heart. When the ultrasonic waves encounter moving blood flow or myocardial tissue in the heart, reflection may occur. After the ultrasound probe receives the reflected ultrasonic waves, the medical device may transmit the reflected ultrasonic waves to the processing device 120. The processing device 120 may calculate the velocity information based on the principle of Doppler shift, and then generate the Doppler ultrasound spectral image. As another example, the processing device 120 may acquire the Doppler ultrasound spectral image that is prestored in the storage device 130, or any other storage device.

In 320, the processing device 120 (e.g., the cardiac cycle determination module 220) may determine a plurality of cardiac cycles based on the Doppler ultrasound spectral image.

As used herein, a cardiac cycle refers to a mechanical activity cycle consisting of one complete contraction and relaxation of the heart. Correspondingly, the cardiac cycle may include a systole and a diastole of the heart. In some embodiments, the cardiac cycle may include phases such as atrial systole, ventricular systole, ventricular diastole, etc.

In some embodiments, the processing device 120 may determine the plurality of cardiac cycles according to the following operations.

In S1021, the processing device 120 may perform, based on the horizontal axis in the Doppler ultrasound spectral image, a segmentation on the Doppler ultrasound spectral image to obtain a first Doppler ultrasound spectral image above the horizontal axis. In other words, the first Doppler ultrasound spectral image may mainly include velocity information associated with the heart during the systole of the heart.

In some embodiments, in a complete cardiac examination record, a Doppler ultrasound spectral image may include a plurality of cardiac cycles and other information that may interfere with analysis. The purpose of the segmentation based on the horizontal axis is to extract the first Doppler ultrasound spectral image above the horizontal axis. As a result, the analysis of subsequent target key points of systole can be more accurate. During the segmentation, the portion below the horizontal axis may also be obtained, which is a second Doppler ultrasound spectral image described below. In other words, the second Doppler ultrasound spectral image may mainly include velocity information associated with the heart during the diastole of the heart. The first Doppler ultrasound spectral image and the second Doppler ultrasound spectral image may not overlap with each other.

In S1022, the processing device 120 may determine a plurality of minimum value positions in an intermediate curve generated based on the first Doppler ultrasound spectral image.

As used herein, the intermediate curve refers to a curve representing velocity changes related to cardiac activity (such as blood flow velocity or myocardial motion velocity) displayed by the Doppler ultrasound spectral image. The minimum value position refers to a position on the horizontal axis (or time axis) where the velocity value reaches a smallest value within a specific local neighborhood.

In some embodiments, the processing device 120 may binarize the first Doppler ultrasound spectral image. The processing device 120 may sum pixel values on the vertical axis of the binarized first Doppler ultrasound spectral image and perform a smoothing process to obtain the intermediate curve. That is, pixel values corresponding to a same position on the horizontal axis may be summed up. The processing device 120 may determine the plurality of minimum value positions based on the intermediate curve.

Merely by way of example, the processing device 120 may convert pixel values in the first Doppler ultrasound spectral image into two values (usually 0 and 1) according to a preset threshold, thereby highlighting main feature contours of cardiac activity velocity information. For example, if a pixel value is smaller than the preset threshold, the processing device 120 may convert the pixel value to value 0, and if a pixel value is greater than or equal to the preset threshold, the processing device 120 may convert the pixel value to value 1. By performing the binarization processing on the first Doppler ultrasound spectral image, the cardiac activity velocity information may be distinguished from the background in the first Doppler ultrasound spectral image. By accumulating the pixel values on the vertical axis of the binarized first Doppler ultrasound spectral image, the vertical feature performance of the cardiac activity velocity information can be enhanced. If a certain position has a similar feature at multiple time points (along the vertical axis direction), the feature may be more obvious after the accumulation.

In some embodiments, the processing device 120 may perform the smoothing processing using a smoothing algorithm. Exemplary smoothing algorithms may include a moving average algorithm, a Gaussian filtering algorithm, a median filtering algorithm, a bilateral filtering algorithm, etc. For example, the processing device 120 may perform the smoothing processing through the moving average algorithm. After the smoothing processing, noise in the first Doppler ultrasound spectral image may be reduced, making the generated intermediate curve smoother, and facilitating accurate identification of the minimum value positions. This is because the Doppler ultrasound spectral image may have small fluctuations due to factors such as equipment signal interference, and the smoothing processing can eliminate these interferences.

In some embodiments, after obtaining the intermediate curve, the processing device 120 may determine the plurality of minimum value positions using an image recognition algorithm. Exemplary image recognition algorithms may include an extremum detection algorithm, a template matching algorithm, a thresholding algorithm, etc. For example, the processing device 120 may compare adjacent pixel summed values on the intermediate curve to identify a specific point where the velocity value reaches a local minimum. The processing device 120 may determine a position of the specific point as a minimum value position.

In S1023, the processing device 120 may determine the plurality of cardiac cycles based on the plurality of minimum value positions.

In some embodiments, since the cardiac activity is a continuous process, the velocity information associated with the heart may exhibit a certain periodic pattern in the Doppler ultrasound spectral image. Thus, the minimum value positions may correspond to key nodes in the cardiac activity, and a portion between two adjacent minimum value positions may be highly likely to contain a complete cardiac cycle. Therefore, the cardiac cycles may be divided by cyclically traversing the plurality of minimum value positions.

In some embodiments, the processing device 120 may determine the plurality of cardiac cycles using a trained machine learning model. Merely by way of example, the processing device 120 may input the first Doppler ultrasound spectral image into the trained machine learning model. Then, the trained machine learning model may output the plurality of cardiac cycles. The trained machine learning model may be obtained by training using a plurality of training samples. Each training sample may include a sample first Doppler ultrasound spectral image. The sample first Doppler ultrasound spectral image is a portion of a sample Doppler ultrasound spectral image above a horizontal axis in the sample Doppler ultrasound spectral image. Each training sample may further include a plurality of sample cardiac cycles corresponding to the sample Doppler ultrasound spectral image. During the training of the trained machine learning model, the plurality of sample cardiac cycles corresponding to the sample Doppler ultrasound spectral image may be used as a label.

According to some embodiments of the present disclosure, by determining the cardiac cycles based on the Doppler ultrasound spectral image, it is possible to eliminate reliance on electrocardiograms (ECGs). Thus, doctors no longer need to attach ECG electrodes to patients before each examination, which saves time and costs and is more in line with doctors' clinical habits.

In 330, the processing device 120 (e.g., the evaluation parameter determination module 230) may determine the one or more evaluation parameters for evaluating a function of the heart based on the plurality of cardiac cycles and the Doppler ultrasound spectral image.

As used herein, the evaluation parameters refer to indicators that can reflect the working state of the heart. The evaluation parameters may be used to measure the cardiac systolic function, diastolic function, and overall pumping function from different perspectives. In some embodiments, the evaluation parameters may include a peak systolic tissue velocity S', a peak early diastolic tissue velocity e', a peak late diastolic tissue velocity a', an isovolumic contraction time (IVCT), an ejection time (ET), an isovolumic relaxation time (IVRT), or the like, or any combination thereof.

In some embodiments, the processing device 120 may identify key points in the Doppler ultrasound spectral image using a key point detection model (e.g., a first key point detection model or a second key point detection model) based on the plurality of cardiac cycles and the Doppler ultrasound spectral image. As used herein, the key points in the Doppler ultrasound spectral image refer to points for locating cardiac cycle phases and extracting cardiac function evaluation parameters. In some embodiments, the key points may include a peak point of systolic motion velocity, a start point of an ejection phase, an end point of the ejection phase, a peak point and a start point of early diastolic motion velocity, a peak point and an end point of late diastolic motion velocity, or the like, or any combination thereof. The processing device 120 may determine the evaluation parameters based on the key points. For example, the processing device 120 may determine the evaluation parameters based on positions of the key points (also referred to as key point positions).

According to some embodiments of the present disclosure, by determining the plurality of cardiac cycles based on the Doppler ultrasound spectral image, and further by determining the evaluation parameters based on the plurality of cardiac cycles and the Doppler ultrasound spectral image, the evaluation parameters can be automatically recognized, thereby improving the detection efficiency and enhancing the accuracy of the determined evaluation parameters.

FIG. 3B is a schematic flowchart illustrating an exemplary process 300B for determining one or more evaluation parameters of a heart according to some embodiments of the present disclosure. In some embodiments, the processing device 120 may determine the one or more evaluation parameters according to the following operations.

In S1031, the processing device 120 may divide the first Doppler ultrasound spectral image into a plurality of first sub-images based on the plurality of cardiac cycles. As used herein, the first sub-images refer to small partial images of the first Doppler ultrasound spectral image divided by the plurality of cardiac cycles, and each first sub-image corresponds to a complete cardiac cycle. For example, the portion between timestamps corresponding to two adjacent minimum value positions in the first Doppler ultrasound spectral image or a portion between a timestamp corresponding to a minimum value position and a timestamp corresponding to a start or end position along the horizontal axis of the first Doppler ultrasound spectral image may be determined as a first sub-image. The cardiac cycle corresponding to the first sub-image may also be referred to as a first cardiac cycle. That is, the first cardiac cycle may be a cardiac cycle between two timestamps corresponding to two adjacent minimum value positions.

In S1032, the processing device 120 may input the plurality of first sub-images into a first key point detection model to obtain first target key point positions in the systole within each first cardiac cycle.

Merely by way of example, for each first sub-image of the plurality of first sub-images, the processing device 120 may determine a first key point heatmap set of the first sub-image and a first score of the first sub-image by inputting the first sub-image into the first key point detection model. The first key point heatmap set may include a plurality of heatmaps of the key points (also referred to as key point heatmaps). Each pixel in the heatmap shows a probability that the pixel corresponds to a key point. The first score may reflect a recognition quality (recognition accuracy) of the first target key point in the first sub-image. The greater the first score, the greater the recognition quality of the first target key point in the first sub-image. The first target key point refers to a point for extracting cardiac function evaluation parameters related to the systole. In some embodiments, the first target key point may include a peak point of systolic motion velocity, a start point of an ejection phase, and an end point of the ejection phase, or the like, or any combination thereof.

Correspondingly, the first target key point positions in the systole may be used to describe a motion velocity characteristic of the heart during the systole. In some embodiments, the first target key point positions of the systole may include a peak position of systolic motion velocity, a start position of an ejection phase, and an end position of the ejection phase, or the like, or any combination thereof. The peak position of systolic motion velocity refers to a position on the first Doppler ultrasound spectral image (or the Doppler ultrasound spectral image) where the cardiac motion velocity reaches its maximum value during systole. The start position of the ejection phase is a starting point of the change in motion velocity when the ejection phase begins. The end position of the ejection phase is an end point of the change in motion velocity when the ejection phase ends.

Further, the processing device 120 may determine one or more first target sub-images from the plurality of first sub-images based on first scores of the plurality of first sub-images. For example, the processing device 120 may determine a first sub-image with a first score greater than or equal to a first score threshold as a first target image. The first score threshold may be set according to a default setting of the ultrasound imaging system 100 or by an operator via the terminal device 140.

The processing device 120 may determine the first target key point positions in the systole in each cardiac cycle corresponding to each first target sub-image by post-processing the first key point heatmap set of the first target sub-image. For example, for each first target sub-image, the processing device 120 may perform double upsampling on the key point heatmap set of the first target sub-image to obtain a key point heatmap set with the same size as the first target sub-image. The processing device 120 may identify the heatmaps of the first target key points, and perform Argmax processing on the heatmaps of the first target key points, respectively, to obtain the corresponding first target key point positions and confidence levels in the first target sub-image. The first target key point positions in a heatmap is a position (or pixel) with the greatest possibility that the position (or pixel) corresponds to a first target key point position. The confidence level refers to a possibility of the position (or pixel) that the position (or pixel) corresponds to a first target key point position. In some embodiments, the processing device 120 may display the first target key point positions and the corresponding confidence levels in each first target sub-image to a user (e.g., a doctor).

In some embodiments, the first key point detection model may be trained in advance to identify and locate key points or regions in the input first sub-images. The first key point detection model may be constructed based on a machine learning algorithm (such as a support vector machine algorithm) or a deep learning algorithm (such as a convolutional neural network), and trained with a large amount of annotated data to learn how to accurately find the first target key point positions (or first target key points) related to systolic motion velocity from the input first sub-images.

Merely by way of example, the first key point detection model may include an encoding layer and a key point decoding layer. The encoding layer selects EfficientNetV2-S as a backbone network for feature extraction of the first sub-images. The decoding layer uses UperNet fine-tuning for key point detection. Based on the pre-annotated peak positions of systolic motion velocity, start positions of ejection phases, and end positions of the ejection phases in the sample first Doppler ultrasound spectral image, sample first key point heatmaps are generated for model training. The AdamW optimizer is adopted for model training, and a loss function uses local heatmap loss. After the model training converges, the model with the best performance on a test set is selected as the first key point detection model. In application, the first sub-images are input into the first key point detection model, processed by the encoding layer, then passed through the decoding layer, and the corresponding detection results are output through a peak point convolution layer, a start point convolution layer, and an end point convolution layer.

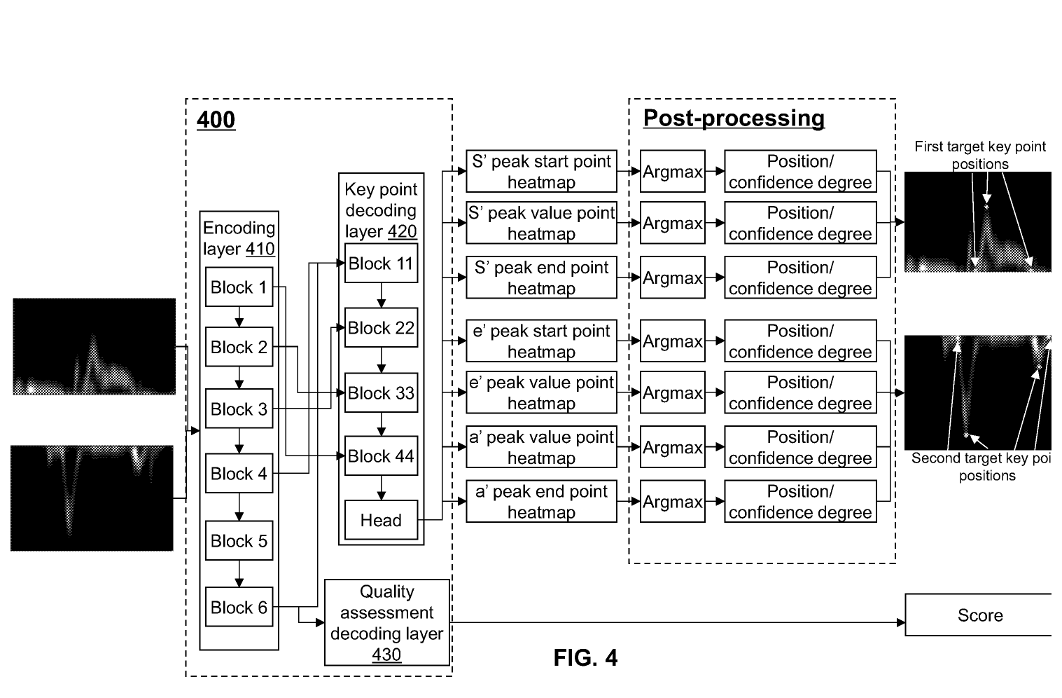

More descriptions about the first key point detection model and the post-processing of the first key point heatmap sets may be found elsewhere in the present disclosure, e.g., FIG. 4 or FIG. 9, and the descriptions thereof.

According to some embodiments of the present disclosure, by dividing the first Doppler ultrasound spectral image into a plurality of first sub-images based on the plurality of first cardiac cycles, and using the first key point detection model to obtain the first target key point positions in the systole within each cardiac cycle, tedious steps of manual measurement can be avoided, thus reducing subjective errors, thereby improving the detection efficiency, enhancing the accuracy of the determined evaluation parameters, and achieving the automation of cardiac function assessment.

In S1033, the processing device 120 may map peak positions of systolic motion velocity, start positions of ejection phases, and end positions of the ejection phases in a plurality of first cardiac cycles to the Doppler ultrasound spectral image. Each of the plurality of first cardiac cycles may be a cardiac cycle corresponding to a first target sub-image. For example, the processing device 120 may arrange the plurality of first sub-images in a chronological order. The processing device 120 may mark the corresponding peak positions of systolic motion velocity, start positions of ejection phases, and end positions of the ejection phases in the plurality of first sub-images on the Doppler ultrasound spectral image to obtain a marked Doppler ultrasound spectral image.

In S1034, the processing device 120 may determine, based on the peak positions of systolic motion velocity corresponding to the plurality of first cardiac cycles, a plurality of second sub-images by performing a period division on a second Doppler ultrasound spectral image that is below the horizontal axis in the Doppler ultrasound spectral image.

As used herein, the second sub-images refer to small partial images divided by the plurality of cardiac cycles, and each second sub-image corresponds to a complete cardiac cycle. For example, the portion between timestamps corresponding to two adjacent peak positions of systolic motion velocity in the second Doppler ultrasound spectral image or a portion between a timestamp corresponding to a peak position of systolic motion velocity and a timestamp corresponding to a start or end position along the horizontal axis of the second Doppler ultrasound spectral image may be determined as a second sub-image. The cardiac cycle corresponding to the second sub-image may also be referred to as a second cardiac cycle. That is, the second cardiac cycle may be a cardiac cycle between two timestamps corresponding to two adjacent peak positions of systolic motion velocity.

In S1035, the processing device 120 may input the plurality of second sub-images into a second key point detection model to obtain second target key point positions in the diastole within each second cardiac cycle.

In some embodiments, the determination manner of the second target key point positions may be similar to the determination manner of the first target key point positions. For example, for each second sub-image, the processing device 120 may determine a second key point heatmap set of the second sub-image and a second score of the second sub-image by inputting the second sub-image into the second key point detection model. The second key point heatmap set may also include a plurality of heatmaps of the key points. The second score may reflect a recognition quality (recognition accuracy) of a second target key point in the second sub-image. The greater the second score, the greater the recognition quality of the second target key point in the second sub-image. The second target key point refers to a point for extracting cardiac function evaluation parameters related to the diastole. In some embodiments, the second target key point may include a peak point and a start point of early diastolic motion velocity, and a peak point and an end point of late diastolic motion velocity, or the like, or any combination thereof. In some embodiments, the first target key points and the second target key points may be collectively referred to as target key points.

Correspondingly, the second target key point positions may be used to describe a motion velocity characteristic of the heart during the diastole. In some embodiments, the second target key point positions of the diastole may include a peak position and a start position of early diastolic motion velocity, and a peak position and an end position of late diastolic motion velocity, or the like, or any combination thereof. During the cardiac cycle of the heart, the early diastole refers to a phase where blood in the atria rapidly fills the ventricles after the ventricles start to relax. The peak position of early diastolic motion velocity is a corresponding point on the Doppler ultrasound spectral image when the motion velocity of the myocardium or related tissues (such as the mitral annulus) reaches its maximum value during this phase. The start position of early diastolic motion velocity refers to a position on the time axis where the early diastolic motion velocity begins to show significant changes (usually starting to rise from a lower velocity). The late diastole refers to a phase where the atria contract to further squeeze the remaining blood into the ventricles. The peak position of the late diastolic motion velocity is a corresponding point on the time axis of the Doppler ultrasound spectral image when the motion velocity of the myocardium or related tissues reaches its maximum value during this phase. The end position of late diastolic motion velocity refers to a position on the time axis where the late diastolic motion velocity stops changing (usually gradually decreasing from a higher velocity to a relatively stable state).

Further, the processing device 120 may determine one or more second target sub-images from the plurality of second sub-images based on second scores of the plurality of second sub-images. For example, the processing device 120 may also determine a second sub-image with a second score greater than or equal to a second score threshold as a second target image. The second score threshold may be set according to a default setting of the ultrasound imaging system 100 or by an operator via the terminal device 140. In some embodiments, the second score threshold may be the same as or different from the first score threshold. The processing device 120 may determine the second target key point positions in the diastole in each cardiac cycle corresponding to each second target sub-image by post-processing the second key point heatmap set of the second target sub-image.

In some embodiments, before inputting the second sub-images into the second key point detection model, the processing device 120 may preprocess the second sub-images. In some embodiments, the preprocessing may include operations such as denoising and standardization to ensure the quality of the input data.

In some embodiments, the second key point detection model may be trained in advance to identify and locate key points or regions in the input second sub-images. The second key point detection model may be constructed based on a machine learning algorithm (such as a support vector machine algorithm) or a deep learning algorithm (such as a convolutional neural network), and trained with a large amount of annotated data to learn how to accurately find the second target key point positions (or second target key points) related to diastolic motion velocity from the input second sub-images. In some embodiments, the second key point detection model and the first key point detection model may be the same or different. When the second key point detection model is the same as the first key point detection model, during the training of the second key point detection model, an input of the second key point detection model may be a sample second sub-image, and an output of the second key point detection model may be the second target key point positions of the diastole; while during the training of the first key point detection model, an input of the first key point detection model may be a sample first sub-image, and an output of the first key point detection model may be the first target key point positions of the systole. In some embodiments, when the second key point detection model is the same as the first key point detection model, both the second key point detection model and the first key point detection model may be referred to as the key point detection model.

According to some embodiments of the present disclosure, by dividing the second Doppler ultrasound spectral image into a plurality of second sub-images based on the plurality of second cardiac cycles, and using the second key point detection model to obtain the second target key point positions in the diastole within each cardiac cycle, tedious steps of manual measurement can be avoided, thus reducing subjective errors, thereby improving the detection efficiency, enhancing the accuracy of the determined evaluation parameters, and achieving the automation of cardiac function assessment.

In some embodiments, for each key point heatmap (e.g., a first key point heatmap or a second key point heatmap) output by the key point detection model, the processing device 120 may determine a count of target pixels of the key point heatmap. A pixel value of each target pixel may be greater than a first preset threshold. The pixel value of each target pixel may reflect a possibility that the target pixel is a key point. The processing device 120 may determine a standard deviation of counts of target pixels of key point heatmaps of a plurality of sub-images (e.g., the first sub-images or the second sub-images). The standard deviation may reflect a blur degree of the sub-image. The greater the standard deviation, the more blurry the sub-image.

In response to the standard deviation being greater than a second preset threshold, the processing device 120 may reacquire a Doppler ultrasound spectral image of the heart. For example, the processing device 120 may send a prompt to the terminal device 140 (or a display device) to remind the staff to re-collect a Doppler ultrasound spectral image. As another example, the processing device 120 may directly control the medical device 110 to re-collect a Doppler ultrasound spectral image. In some embodiments, the first preset threshold and the second preset threshold may be set based on user experience.

According to some embodiments of the present disclosure, by analyzing the blurriness of the heatmaps output by the key point detection model, a new Doppler ultrasound spectral image can be recollected in a timely manner when the key point detection model output is unclear, ensuring the quality of the input data at the source.

In S1036, the processing device 120 may determine a target Doppler ultrasound spectral image by mapping the peak positions and start positions of early diastolic motion velocity, and peak positions and end positions of late diastolic motion velocity in a plurality of second cardiac cycles to the marked Doppler ultrasound spectral image. In other words, all target key points including the peak positions of systolic motion velocity, the start positions and the end positions of the ejection phase, peak positions and start positions of early diastolic motion velocity, and peak positions and end positions of late diastolic motion velocity in the plurality of second cardiac cycles may be mapped back to the Doppler ultrasound spectral image to determine the target Doppler ultrasound spectral image. Each of the plurality of second cardiac cycles may be a cardiac cycle corresponding to a second target sub-image. For example, the processing device 120 may arrange the plurality of second sub-images in a chronological order. The processing device 120 may mark the corresponding peak positions and start positions of early diastolic motion velocity, and peak positions and end positions of late diastolic motion velocity in the plurality of second sub-images on the marked Doppler ultrasound spectral image to obtain the target Doppler ultrasound spectral image. That is, the target Doppler ultrasound spectral image is a Doppler ultrasound spectral image marked with the key points.

According to some embodiments of the present disclosure, by mapping the first and second target key point positions to the Doppler ultrasound spectral image to obtain the target Doppler ultrasound spectral image, the quantitative extraction of the evaluation parameters can be achieved, and the subjective errors of manual interpretation can be avoided, thereby improving the analysis accuracy, and supporting the automated diagnosis and standardized evaluation.

In S1037, the processing device 120 may determine the one or more evaluation parameters based on the target Doppler ultrasound spectral image.

In some embodiments, the processing device 120 may determine a velocity corresponding to a peak position of systolic motion velocity in the target Doppler ultrasound spectral image as the peak systolic tissue velocity S'. In some embodiments, the processing device 120 may determine a velocity corresponding to a peak position of early diastolic motion velocity in the target Doppler ultrasound spectral image as the peak early diastolic tissue velocity e'. In some embodiments, the processing device 120 may determine a velocity corresponding to a peak position of late diastolic motion velocity in the target Doppler ultrasound spectral image as the peak late diastolic tissue velocity a'. In some embodiments, the processing device 120 may determine a time from an end position of late diastolic motion velocity to a start position of an ejection phase adjacent on a right side in the target Doppler ultrasound spectral image as the IVCT. In some embodiments, the processing device 120 may determine a time between the start position and the end position of the ejection phase in the target Doppler ultrasound spectral image as the ejection time. In some embodiments, the processing device 120 may determine a time from the end position of the ejection phase to a start position of early diastolic motion velocity of an early diastole adjacent on a right side in the target Doppler ultrasound spectral image as the IVRT.

In some embodiments, a user (e.g., a doctor) may select several of the one or more evaluation parameters to assess the heart.

In some embodiments, the processing device 120 may determine a target peak systolic tissue velocity S'’ by calculating an average value of peak systolic tissue velocities S' in the plurality of cardiac cycles, or by determining a peak systolic tissue velocity S' in one cardiac cycle based on a first selection instruction issued by the user. In some embodiments, the processing device 120 may determine a target peak early diastolic tissue velocity e'’ by calculating an average value of peak early diastolic tissue velocities e' in the plurality of cardiac cycles, or by determining a peak early diastolic tissue velocity e' in one cardiac cycle based on a second selection instruction issued by the user. In some embodiments, the processing device 120 may determine a target peak late diastolic tissue velocity a'’ by calculating an average value of peak late diastolic tissue velocities a' in the plurality of cardiac cycles, or by determining a peak late diastolic tissue velocity a' in one cardiac cycle based on a third selection instruction issued by the user. In some embodiments, the processing device 120 may determine a target IVCT by calculating an average value of IVCTs in the plurality of cardiac cycles, or by determining an IVCT in one cardiac cycle based on a fourth selection instruction issued by the user. In some embodiments, the processing device 120 may determine a target ejection time by calculating an average value of ejection times in the plurality of cardiac cycles, or by determining an ejection time in one cardiac cycle based on a fifth selection instruction issued by the user. In some embodiments, the processing device 120 may determine a target IVRT by calculating an average value of IVRTs in the plurality of cardiac cycles, or by determining an IVRT in one cardiac cycle based on a sixth selection instruction issued by the user.

In some embodiments, the processing device 120 may calculate a standard deviation of values of a target parameter in the plurality of second cardiac cycles. The target parameter may be at least one of the one or more evaluation parameters. For example, the target parameter may be the peak systolic tissue velocity S', the peak early diastolic tissue velocity e', the peak late diastolic tissue velocity a', the IVCT, the ET, or the IVRT.

The processing device 120 may select a plurality of target cardiac cycles from the plurality of second cardiac cycles based on the standard deviation of the target parameter. For example, the processing device 120 may calculate an average value and a standard deviation of values of a specific target parameter (e.g., the peak systolic tissue velocity S') of the plurality of second cardiac cycles. The processing device 120 may treat cardiac cycles that deviate from the average value by more than 2 standard deviations as outliers, and exclude them from the available data range, and the remaining cardiac cycles are the target cardiac cycles. The processing device 120 may only display the data associated with the target cardiac cycles to the user.

According to some embodiments of the present disclosure, automatically eliminating cardiac cycles with abnormal parameters through statistical techniques improves the stability and credibility of the finally calculated average values of the evaluation parameters.

In some embodiments, the processing device 120 may designate the peak systolic tissue velocity S' in a cardiac cycle with a code input by the user as the target peak systolic tissue velocity S'’, or designate a value of the peak systolic tissue velocity S' among values of the peak systolic tissue velocity S' in the plurality of cardiac cycles as the target peak systolic tissue velocity S'’. In some embodiments, the processing device 120 may designate the peak early diastolic tissue velocity e' in a cardiac cycle with a code input by the user as the target peak early diastolic tissue velocity e'’, or designate a value of the peak early diastolic tissue velocity e' among values of the peak early diastolic tissue velocity e' in the plurality of cardiac cycles as the target peak early diastolic tissue velocity e'’. In some embodiments, the processing device 120 may designate the peak late diastolic tissue velocity a' in a cardiac cycle with a code input by the user as the target peak late diastolic tissue velocity a'’, or designate a value of the peak late diastolic tissue velocity a' among values of the peak late diastolic tissue velocity a' in the plurality of cardiac cycles as the target peak late diastolic tissue velocity a'’. In some embodiments, the processing device 120 may designate the IVCT in a cardiac cycle with a code input by the user as the target IVCT, or designate a value of the IVCT among values of the IVCT in the plurality of cardiac cycles as the target IVCT. In some embodiments, the processing device 120 may designate the ejection time in a cardiac cycle with a code input by the user as the target ejection time, or designate a value of the ejection time among values of the ejection time in the plurality of cardiac cycles as the target ejection time. In some embodiments, the the processing device 120 may designate the IVRT in a cardiac cycle with a code input by the user as the target IVRT, or designate a value of the IVRT among values of the IVRT in the plurality of cardiac cycles as the target IVRT.

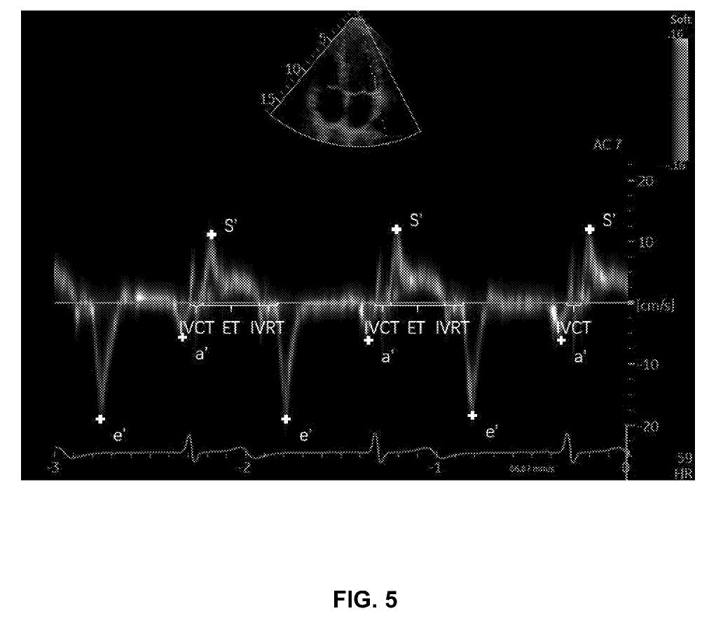

In some embodiments, the processing device 120 may mark the one or more evaluation parameters on the target Doppler ultrasound spectral image for display to the user, as shown in FIG. 5. In some embodiments, the user (e.g., an experienced doctor) may adjust the one or more evaluation parameters via a terminal device (e.g., the terminal device 140).

It should be noted that the above description is merely provided for the purposes of illustration and is not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be omitted and/or one or more additional operations may be added. For example, operation 310 and operation 320 may be combined into a single operation. As another example, one or more other optional operations (e.g., a reconstruction operation) may be added.

FIG. 4 is a schematic diagram illustrating determining target key point positions and scores of sub-images using an exemplary key point detection model 400 according to some embodiments of the present disclosure. The key point detection model 400 may be an exemplary first key point detection model or an exemplary second key point detection model as described in connection with FIG. 1. An input of the key point detection model 400 may include a sub-image (e.g., a first sub-image or a second sub-image), and an output of the key point detection model 400 may include a key point heatmap set of the sub-image and a score of the sub-image. As shown in FIG. 4, the key point detection model 400 may include an encoding layer 410, a key point decoding layer 420, and a quality assessment decoding layer 430. Both the key point decoding layer 420 and the quality assessment decoding layer 430 may be connected to the encoding layer 410.

The encoding layer 410 may be configured to extract the feature maps of the inputted sub-image. An input of the encoding layer 410 may include a sub-image, and an output of the encoding layer 410 may include feature maps of the sub-image. In some embodiments, the encoding layer 410 may adopt a MobileNetV4-ConvSmall structure as a backbone network for feature extraction of sub-images. In some embodiments, the encoding layer 410 may include a plurality of blocks, such as block 1, block 2, block 3, block 4, block 5, and block 6.

Merely by way of example, the block 1 may include one convbn layer. An input of the block 1 is a specific sub-image (e.g., a first sub-image or a second sub-image), and an output of the block 1 is a first feature map of the specific sub-image. In the present disclosure, one convbn layer may be composed of a 2D convolutional layer (conv2d), a BatchNorm2d layer, and a ReLU layer. The block 2 may include two convbn layers. An input of the block 2 is the first feature map, and an output of the block 2 is a second feature map of the specific sub-image. The block 3 may include two convbn layers. An input of the block 3 is the second feature map, and an output of the block 3 is a third feature map of the specific sub-image. The block 4 may include six uib layers, such as a first uib layer 1, a second uib layer 1, a third uib layer 1, a fourth uib layer 1, a fifth uib layer 1, and a sixth uib layer 1. In some embodiments, each uib layer may include a plurality of convbn layers, e.g., 3, 4, 5, etc. For example, the first uib layer 1 may include four convbn layers. Each of the second uib layer 1, the third uib layer 1, the fourth uib layer 1, the fifth uib layer 1, and the sixth uib layer 1 may include three convbn layers. An input of the block 4 is the third feature map, and an output of the block 4 is a fourth feature map of the specific sub-image. The block 5 may include six uib layers, such as a first uib layer 2, a second uib layer 2, a third uib layer 2, a fourth uib layer 2, a fifth uib layer 2, and a sixth uib layer 2. Both the first uib layer 2 and the second uib layer 2 may include four convbn layers. Each of the third uib layer 2, the fourth uib layer 2, the fifth uib layer 2, and the sixth uib layer 2 may include three convbn layers. An input of the block 5 is the fourth feature map, and an output of the block 1 is a fifth feature map of the specific sub-image. The block 6 may include two convbn layers. An input of the block 6 is the fifth feature map, and an output of the block 6 is a sixth feature map of the specific sub-image.

The key point decoding layer 420 may be configured to generate key point heatmap set of the sub-image. An input of the key point decoding layer 420 may include the feature maps of the sub-image, and an output of the key point decoding layer 420 may include the key point heatmap set of the sub-image. In some embodiments, the key point decoding layer 420 may adopt a ResUnet network for feature decoding. In some embodiments, the key point decoding layer 420 may include a plurality of blocks, such as block 11, block 22, block 33, block 44, and a head layer. Merely by way of example, each of the block 11, block 22, block 33, and block 44 may include an attention module and a ResNet block, and the head layer may include three convbn layers. An input of the block 11 is the fourth feature map and the sixth feature map, and an output of the block 11 is a first fusion map. An input of the block 22 is the third feature map and the first fusion map, and an output of the block 22 is a second fusion map. An input of the block 33 is the second feature map and the second fusion map, and an output of the block 33 is a third fusion map. An input of the block 44 is the first feature map and the third fusion map, and an output of the block 44 is a fourth fusion map. An input of the head layer is the fourth fusion map, and an output of the head layer is a key point heatmap set. For example, if the specific sub-image is a first sub-image, the key point heatmap set may be a first key point heatmap set. If the specific sub-image is a second sub-image, the key point heatmap set may be a second key point heatmap set. In some embodiments, the key point heatmap set may include an S’ peak start point heatmap (i.e., a heatmap of the start point of the ejection phase), an S’ peak value point heatmap (i.e., a heatmap of the peak point of systolic motion velocity), an S’ peak end point heatmap (i.e., a heatmap of the end point of the ejection phase), an e’ peak start point heatmap (i.e., a heatmap of the start point of early diastolic motion velocity), an e’ peak value point heatmap (i.e., a heatmap of the peak point of early diastolic motion velocity), an a’ peak value point heatmap (i.e., a heatmap of the peak point of late diastolic motion velocity), and an a’ peak end point heatmap (i.e., a heatmap of the end point of late diastolic motion velocity).

The quality assessment decoding layer 430 may be configured to generate a score of the sub-image. An input of the quality assessment decoding layer 430 may include the feature maps of the sub-image, and an output of the quality assessment decoding layer 430 may include the score of the sub-image. In some embodiments, the quality assessment decoding layer 430 may sequentially include an average pooling layer, a flatten layer, a Linear layer, a ReLU layer, and a Linear layer. An input of the quality assessment decoding layer 430 is the sixth feature map, and an output of the quality assessment decoding layer 430 is a score (e.g., a first score or a second score) for assessing the recognition quality of a target key point in the specific sub-image.

After the key point decoding layer 420 outputs the key point heatmap set, the key point heatmap set may be further post-processed to obtain the corresponding target key point positions. Then, the target key point positions may be mapped back to the corresponding sub-images and/or the Doppler ultrasound spectral image for display to the user. More description about the post-processing may be found elsewhere in the present disclosure, e.g., FIGS. 3A or 3B and the descriptions thereof.

FIG. 5 shows a measurement result according to some embodiments of the present disclosure. As shown in FIG. 5, the one or more evaluation parameters are displayed on the Doppler ultrasound spectral image. The one or more evaluation parameters include a peak systolic tissue velocity S', a peak early diastolic tissue velocity e', a peak late diastolic tissue velocity a', an isovolumic contraction time (IVCT), an ejection time (ET), and an isovolumic relaxation time (IVRT).

According to some embodiments of the present disclosure, measurements can be automatically completed without doctors manually clicking on the peak position and time interval range, which greatly improves the doctors' examination efficiency while enhancing the objectivity of the measurement results. Meanwhile, compared with manual measurement, which usually only provides measurement results for one cardiac cycle, the evaluation parameter determination method provided by some embodiments of the present disclosure can generate measurement results for all cardiac cycles in the Doppler ultrasound spectral image, making the final measured values more reasonable.

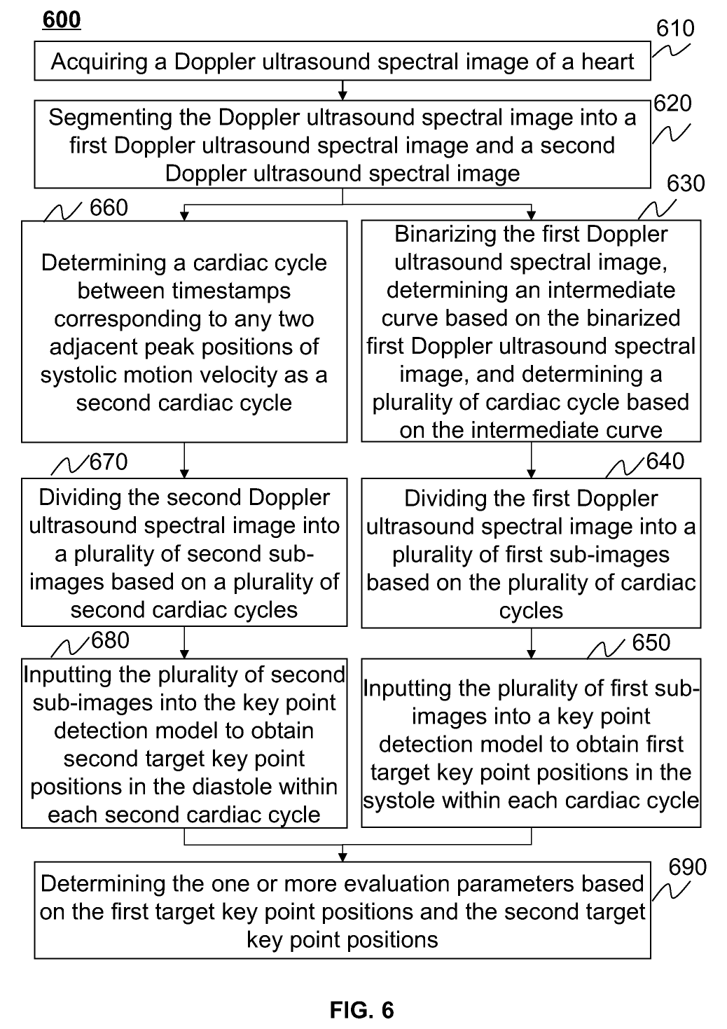

FIG. 6 is a schematic flowchart illustrating an exemplary process 600 for determining one or more evaluation parameters of a heart according to some embodiments of the present disclosure. In some embodiments, the process 600 may be executed by the ultrasound imaging system 100. For example, the process 600 may be implemented as a set of instructions (e.g., an application) stored in the storage device 130, and the processing device 120 (e.g., the modules in FIG. 2A) may execute the set of instructions and may accordingly be directed to perform the process 600. The operations of the illustrated process presented below are intended to be illustrative. In some embodiments, the process 600 may be accomplished with one or more additional operations not described and/or without one or more of the operations discussed. Additionally, the order of the operations of the process 600 illustrated in FIG. 6 and described below is not intended to be limiting.

In 610, the processing device 120 (e.g., the obtaining module 210) may acquire a Doppler ultrasound spectral image of the heart. More descriptions about the acquisition of the Doppler ultrasound spectral image may be found elsewhere in the present disclosure, e.g., operation 310 and the descriptions thereof.

In 620, the processing device 120 (e.g., the cardiac cycle determination module 220) may segment the Doppler ultrasound spectral image into a first Doppler ultrasound spectral image and a second Doppler ultrasound spectral image. The first Doppler ultrasound spectral image is a portion above the horizontal axis of the Doppler ultrasound spectral image, and the second Doppler ultrasound spectral image is a portion below the horizontal axis of the Doppler ultrasound spectral image. More descriptions for segmenting the Doppler ultrasound spectral image may be found elsewhere in the present disclosure, e.g., operation 320 or 330 and the descriptions thereof.

In 630, the processing device 120 (e.g., the cardiac cycle determination module 220) may binarize the first Doppler ultrasound spectral image, determine an intermediate curve based on the binarized first Doppler ultrasound spectral image, and determine a plurality of cardiac cycles based on the intermediate curve. More descriptions about the determination of the intermediate curve and the plurality of cardiac cycles may be found elsewhere in the present disclosure, e.g., FIGS. 3A and 3B and the descriptions thereof.

In 640, the processing device 120 (e.g., the evaluation parameter determination module 230) may divide the first Doppler ultrasound spectral image into a plurality of first sub-images based on the plurality of cardiac cycles. A cardiac cycle corresponding to the first sub-image may also be referred to as a first cardiac cycle. More descriptions for dividing the first Doppler ultrasound spectral image may be found elsewhere in the present disclosure, e.g., operation 320 and the descriptions thereof.

In 650, the processing device 120 (e.g., the evaluation parameter determination module 230) may input the plurality of first sub-images into a key point detection model to obtain first target key point positions in the systole within each first cardiac cycle. In some embodiments, the first target key point positions may include a peak position of systolic motion velocity, a start position of an ejection phase, and an end position of the ejection phase, or the like, or any combination thereof. More descriptions for determining the first target key point positions using the key point detection model may be found elsewhere in the present disclosure, e.g., FIGS. 3A, 3B, and FIG. 4 and the descriptions thereof.

In 660, the processing device 120 (e.g., the evaluation parameter determination module 230) may determine a cardiac cycle between timestamps corresponding to any two adjacent peak positions of systolic motion velocity as a second cardiac cycle.

In 670, the processing device 120 (e.g., the evaluation parameter determination module 230) may divide the second Doppler ultrasound spectral image into a plurality of second sub-images based on a plurality of second cardiac cycles. A second cardiac cycle may correspond to a second sub-image. More descriptions for dividing the second Doppler ultrasound spectral image may be found elsewhere in the present disclosure, e.g., operation 330 and the descriptions thereof.

In 680, the processing device 120 (e.g., the evaluation parameter determination module 230) may input the plurality of second sub-images into the key point detection model to obtain second target key point positions in the diastole within each second cardiac cycle. In some embodiments, the second target key point positions may include a peak position and a start position of early diastolic motion velocity, and a peak position and an end position of late diastolic motion velocity, or the like, or any combination thereof. More descriptions for determining the second target key point positions using the key point detection model may be found elsewhere in the present disclosure, e.g., FIGS. 3A, 3B and FIG. 4 and the descriptions thereof.

In 690, the processing device 120 (e.g., the evaluation parameter determination module 230) may determine the one or more evaluation parameters based on the first target key point positions and the second target key point positions. More descriptions for determining the one or more evaluation parameters may be found elsewhere in the present disclosure, e.g., FIGS. 3A and 3B and the descriptions thereof.

It should be noted that the above description is merely provided for the purposes of illustration and is not intended to limit the scope of the present disclosure. For persons having ordinary skills in the art, multiple variations and modifications may be made under the teachings of the present disclosure. However, those variations and modifications do not depart from the scope of the present disclosure.

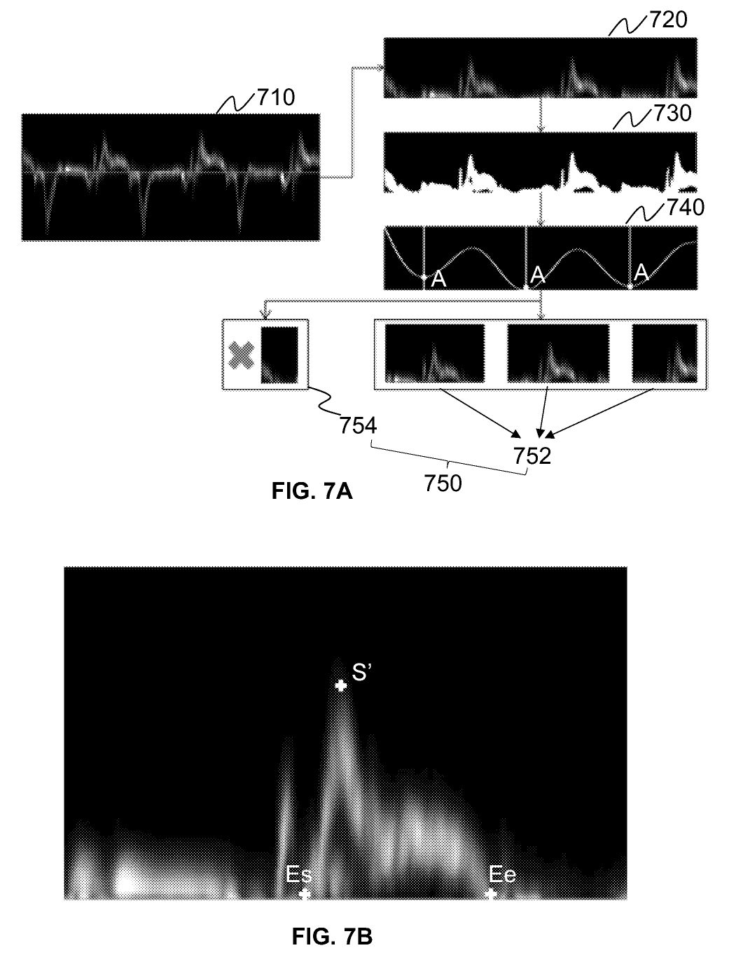

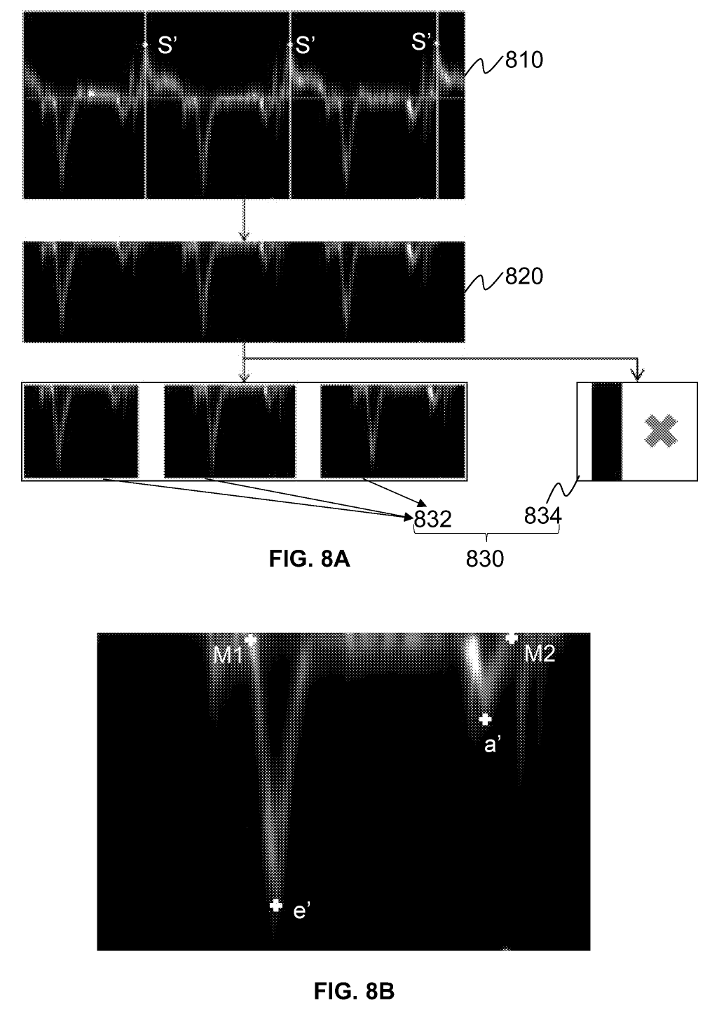

FIG. 7A is a schematic flowchart illustrating an exemplary process for determining a plurality of first sub-images according to some embodiments of the present disclosure. FIG. 7B is a schematic diagram illustrating a first sub-image marked with first target key points according to some embodiments of the present disclosure. As shown in FIG. 7A, a Doppler ultrasound spectral image 710 of a heart may be segmented into a first Doppler ultrasound spectral image 720 and a second Doppler ultrasound spectral image (e.g., the image 820 shown in FIG. 8) based on the horizontal axis of the Doppler ultrasound spectral image 710. The first Doppler ultrasound spectral image 720 may be binarized to generate a binarized first Doppler ultrasound spectral image 730. Pixel values on each vertical axis of the binarized first Doppler ultrasound spectral image 730 may be summed up. Then, the obtained image may be smoothed to obtain an intermediate curve 740. A plurality of minimum value positions A in the intermediate curve 740 may be identified. The first Doppler ultrasound spectral image 720 may be divided into a plurality of first sub-images 750 based on the plurality of minimum value positions A. For example, a portion between timestamps corresponding to any two minimum value positions A in the first Doppler ultrasound spectral image 720 or a portion between a timestamp corresponding to a minimum value position A and a timestamp corresponding to a start or end position along the horizontal axis of the first Doppler ultrasound spectral image may be determined as a first sub-image 750. A cardiac cycle corresponding to the first sub-image 750 may also be referred to as a first cardiac cycle.

The plurality of first sub-images 750 may be input into a key point detection model (e.g., a first key point detection model) to obtain first target key point positions in the systole within each first cardiac cycle. For example, for each first sub-image 750, after inputting the first sub-image 750 into the key point detection model, the key point detection model may output a first key point heatmap set and a first score of the first sub-image 750. One or more first target sub-images may be determined from the plurality of first sub-images 750 based on first scores of the plurality of first sub-images 750. For example, if a first score of a first sub-image is greater than or equal to a first score threshold, the first sub-image may be determined as a first target sub-image, for example, the first sub-images 752 may be determined as first target sub-images. If a first score of a first sub-image is smaller than the first score threshold, the first sub-image may be eliminated, for example, the first sub-image 754 may be eliminated. The first target key point positions in the systole in each first cardiac cycle corresponding to each first target sub-image may be determined by post-processing the first key point heatmap set of the first target sub-image. The first target key point positions may be mapped back to the first sub-image or the Doppler ultrasound spectral image. As shown in FIG. 7B, point S’ represents a peak point of systolic motion velocity, point Es represents a start point of the ejection phase, and point Ee represents an end point of the ejection phase.

FIG. 8A is a schematic flowchart illustrating an exemplary process for determining a plurality of second sub-images according to some embodiments of the present disclosure. FIG. 8B is a schematic diagram illustrating a second sub-image marked with second target key points according to some embodiments of the present disclosure. As shown in FIG. 8A, the first target key point positions in the systole in each first sub-image 750 may be mapped back to the Doppler ultrasound spectral image 710 to generate a marked Doppler ultrasound spectral image 810. A portion below the horizontal axis in the Doppler ultrasound spectral image 810 may be determined as a second Doppler ultrasound spectral image 820. The second Doppler ultrasound spectral image 820 may be divided into a plurality of second sub-images 830 based on the plurality of peak positions S’ of systolic motion velocity in the marked Doppler ultrasound spectral image 810. For example, a portion between timestamps corresponding to any two peak positions S’ of systolic motion velocity in the second Doppler ultrasound spectral image 820 or a portion between a timestamp corresponding to a peak position S’ and a timestamp corresponding to a start or end position along the horizontal axis of the second Doppler ultrasound spectral image may be determined as a second sub-image 830. A cardiac cycle corresponding to the second sub-image may also be referred to as a second cardiac cycle.

The plurality of second sub-images 830 may be input into a key point detection model (e.g., a second key point detection model) to obtain second target key point positions in the diastole within each second cardiac cycle. For example, for each second sub-image 830, after inputting the second sub-image 830 into the key point detection model, the key point detection model may output a second key point heatmap set and a second score of the second sub-image 830. One or more second target sub-images may be determined from the plurality of second sub-images 830 based on second scores of the plurality of second sub-images 830. For example, if a second score of a second sub-image is greater than or equal to a second score threshold, the second sub-image may be determined as a second target sub-image, for example, the second sub-images 832 may be determined as second target sub-images. If a second score of a second sub-image is smaller than the second score threshold, the second sub-image may be eliminated, for example, the second sub-image 834 may be eliminated. The second target key point positions in the diastole in each second cardiac cycle, corresponding to each second target sub-image, may be determined by post-processing the second key point heatmap set of the second target sub-image. The second target key point positions may be mapped back to the second sub-image or the Doppler ultrasound spectral image. As shown in FIG. 8B, point M1 represents a start point of early diastolic motion velocity, e’ represents a peak point of early diastolic motion velocity, point a’ represents a peak point of late diastolic motion velocity, and point M2 represents an end point of late diastolic motion velocity.

FIG. 9 is a schematic flowchart illustrating an exemplary training process 900 of a key point detection model according to some embodiments of the present disclosure. In some embodiments, the process 900 may be executed by the ultrasound imaging system 100. For example, the process 900 may be implemented as a set of instructions (e.g., an application) stored in the storage device 130, and the processing device 120 (e.g., the modules in FIG. 2B) may execute the set of instructions and may accordingly be directed to perform the process 900. The operations of the illustrated process presented below are intended to be illustrative. In some embodiments, the process 900 may be accomplished with one or more additional operations not described and/or without one or more of the operations discussed. Additionally, the order of the operations of the process 900 illustrated in FIG. 9 and described below is not intended to be limiting.

In some embodiments, the first key point detection model or the second key point detection model described in connection with operation 330 in FIGS. 3A or 3B may be obtained according to the process 900. In some embodiments, the process 900 may be performed by another device or system other than the ultrasound imaging system 100, e.g., a device or system of a vendor of a manufacturer. For illustration purposes, the implementation of the process 900 by the processing device 120 is described as an example.

In 910, the processing device 120 (e.g., the obtaining module 240) may obtain a plurality of training samples. The plurality of training samples may include a first training sample set, a second training sample set, a third training sample set, and a fourth training sample set. The first training sample set may include a plurality of sample first sub-images with first labels. The second training sample set may include a plurality of sample second sub-images with second labels. The third training sample set may include a plurality of complete sample first sub-images and a plurality of incomplete sample first sub-images with third labels. The fourth training sample set may include a plurality of complete sample second sub-images and a plurality of incomplete sample second sub-images with fourth labels.

The first labels of the first training sample set may include reference first key point heatmap sets generated based on sample first target key point positions of the plurality of sample first sub-images. The second labels of the second training sample set may include reference second key point heatmap sets generated based on sample second target key point positions of the plurality of sample second sub-images. The third labels of the third training sample set may include reference first scores of the plurality of complete sample first sub-images or reference first scores of the plurality of incomplete sample first sub-images. The fourth labels of the fourth training sample set may include reference second scores of the plurality of complete sample second sub-images or reference second scores of the plurality of incomplete sample second sub-images. It should be noted that the first labels, the second labels, the third labels, and the fourth labels may be manually annotated by experienced doctors.

In 920, the processing device 120 (e.g., the model determination module 250) may generate the key point detection model by training an initial key point detection model using the plurality of training samples. In some embodiments, the initial key point detection model may include a machine learning model, such as a deep learning model, a neural network model, etc. For example, the initial key point detection model may be trained based on the plurality of training samples using a machine learning algorithm.

In some embodiments, the key point detection model may be obtained by performing an iterative operation including a plurality of iterations to iteratively update parameter values of the initial key point detection model. For example, the processing device 120 may perform the plurality of iterations on the initial key point detection model based on a target loss function using the plurality of training samples until a convergence condition is reached. In each iteration, an input of the initial key point detection model (or an updated initial key point detection model) may include a portion of sample first sub-images, a portion of sample second sub-images, a portion of complete sample first sub-images and incomplete sample first sub-images, and a portion of complete sample second sub-images and incomplete sample second sub-images with fourth labels. An output of the initial key point detection model (or the updated initial key point detection model) may include a sample first key point heatmap set corresponding to the portion of sample first sub-images, a sample second key point heatmap set corresponding to the portion of sample second sub-images, a sample first score corresponding to the portion of complete sample first sub-images and incomplete sample first sub-images, and a sample second score corresponding to the portion of complete sample second sub-images and incomplete sample second sub-images. It should be noted that a same set or different sets of training samples may be used in different iterations in training the initial key point detection model.

In some embodiments, the target loss function may include a first loss term, a second loss term, a third loss term, and a fourth loss term. The first loss term may reflect a positioning accuracy of a target key point position in the systole. The second loss term may reflect a positioning accuracy of a target key point position in a diastole. The third loss term may reflect an accuracy of quality assessment of each sample first sub-image. The fourth loss term may reflect an accuracy of quality assessment of each sample second sub-image.

In some embodiments, the processing device 120 may determine a value of the target loss function by weighting and summing the first loss term, the second loss term, the third loss term, and the fourth loss term. For example, in the current iteration, a sample first sub-image, a sample second sub-image, a complete sample first sub-image (or an incomplete sample first sub-image), and a complete sample second sub-image (or an incomplete sample second sub-image) are input into the updated initial key point detection model. For the sample first sub-image, the updated initial key point detection model may output a sample first key point heatmap set. The processing device 120 may determine the first loss term based on the sample first key point heatmap set and the corresponding reference first key point heatmap set. For the sample second sub-image, the updated initial key point detection model may output a sample second key point heatmap set. The processing device 120 may determine the second loss term based on the sample second key point heatmap set and the corresponding reference second key point heatmap set. For the complete sample first sub-image (or the incomplete sample first sub-image), the updated initial key point detection model may output a sample first score. The processing device 120 may determine the third loss term based on the sample first score and the corresponding reference first score. For the complete sample second sub-image (or the incomplete sample second sub-image), the updated initial key point detection model may output a sample second score. The processing device 120 may determine the fourth loss term based on the sample second score and the corresponding reference second score. The processing device 120 may determine a value of the target loss function by weighting and summing the first loss term, the second loss term, the third loss term, and the fourth loss term.

The processing device 120 may perform weighted summation on the first loss term, the second loss term, the third loss term, and the fourth loss term based on a first weight corresponding to the first loss term, a second weight corresponding to the second loss term, a third weight corresponding to the third loss term, and a fourth weight corresponding to the fourth loss term. A sum of the first weight, the second weight, the third weight, and the fourth weight may be equal to 1. For example, both the first weight and the second weight may be 0.4, and both the third weight and the fourth weight may be 0.1. In some embodiments, the first weight, the second weight, the third weight, and the fourth weight may be set according to experience.

In some embodiments, the first weight and the second weight, the third weight, and the fourth weight may be determined according to a convergence speed corresponding to each loss term. Each loss term may correspond to a task. The convergence speed may reflect a difficulty level of the corresponding task. For example, if the convergence speed is fast, it indicates that the update key point detection model is learning the task smoothly, and the corresponding task difficulty level is low (easy); otherwise, the corresponding task difficulty level is high (difficult).

In some embodiments, the processing device 120 may determine the convergence speed of each loss term during one or more preceding rounds of training. The convergence speed refers to a descending speed of each type of loss term in one iteration or an average descending speed over multiple iterations. For example, the processing device 120 may determine the convergence speed of each loss term by using the value of the loss term in the previous iteration minus the value of the loss term in the current iteration. The processing device 120 may determine a weight corresponding to each loss term based on the convergence speed of each loss term. For example, if the corresponding convergence speed is greater than a first preset speed threshold, the processing device 120 may reduce the corresponding weight by a preset ratio (e.g., 5%, 7%, 10%, etc.). If the corresponding convergence speed is less than a second preset speed threshold, the processing device 120 may increase the corresponding weight by the preset ratio. The first preset speed threshold and the second preset speed threshold may be set based on experience. The first preset speed threshold may be greater than the second preset speed threshold.

According to some embodiments of the present disclosure, by dynamically adjusting loss weights according to the learning difficulty level of different tasks, the training process can be intelligently balanced, thereby accelerating the convergence speed of the key point detection model and improving the training effect.

In some embodiments, the target loss function may further include a fifth loss term. The fifth loss term may reflect a local consistency and a unimodality of a predicted key point. For example, the processing device 120 may binarize the key point heatmap generated by the key point detection model (for example, take the region with a probability > 0.75). The processing device 120 may calculate a count of connected components in the binarized image. The processing device 120 may determine an absolute value of a difference between the count of the connected components and one as the fifth loss.

According to some embodiments of the present disclosure, introducing the fifth loss term can effectively suppress the key point detection model from generating multiple scattered "bright spots" during prediction, ensuring the uniqueness and clarity of the key point localization results.

In some embodiments, the convergence condition may be that the value of the target loss function obtained in the current iteration is less than a loss threshold, that a certain count of iterations is performed, that the target loss function converges such that differences of the values of the target loss function obtained in consecutive iterations are within a threshold, etc. If the convergence condition is satisfied in the current iteration, the processing device 120 may designate the updated initial key point detection model as the key point detection model. In some embodiments, the key point detection model may be transmitted to a storage device (e.g., the storage device 130) for storage.

It should be noted that the above description is merely provided for the purposes of illustration and is not intended to limit the scope of the present disclosure. For persons having ordinary skills in the art, multiple variations and modifications may be made under the teachings of the present disclosure. However, those variations and modifications do not depart from the scope of the present disclosure. For example, after the key point detection model is generated, the processing device 120 may further test the key point detection model using a set of testing samples. As another example, the processing device 120 may update the key point detection model periodically or irregularly based on one or more newly-generated training samples. As a further example, an operator (e.g., an engineer) may manually calibrate or update the key point detection model.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer-readable media having computer-readable program code embodied thereon.

A non-transitory computer-readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer-readable signal medium may be any computer-readable medium that is not a computer-readable storage medium and that may communicate, propagate, or transport a program for use by or in connection with an instruction execution system, apparatus, or device. Program code embodied on a computer-readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object-oriented programming language such as Java, Scala, Smalltalk, Eiffel, JADE, Emerald, C++, C#, VB. NET, Python or the like, conventional procedural programming languages, such as the "C" programming language, Visual Basic, Fortran, Perl, COBOL, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method for determining one or more evaluation parameters of a heart, performed by a computing device including at least one processor and at least one storage device, comprising:
obtaining a Doppler ultrasound spectral image of the heart;
determining a plurality of cardiac cycles based on the Doppler ultrasound spectral image; and
determining the one or more evaluation parameters for evaluating a function of the heart based on the plurality of cardiac cycles and the Doppler ultrasound spectral image.

2. The method of claim 1, wherein the determining a plurality of cardiac cycles based on the Doppler ultrasound spectral image includes:
dividing, based on a horizontal axis in the Doppler ultrasound spectral image, the Doppler ultrasound spectral image into a first Doppler ultrasound spectral image and a second Doppler ultrasound spectral image, the first Doppler ultrasound spectral image being a portion of the Doppler ultrasound spectral image above the horizontal axis, the second Doppler ultrasound spectral image being a portion of the Doppler ultrasound spectral image below the horizontal axis; and
determining the plurality of cardiac cycles based on the first Doppler ultrasound spectral image and the second Doppler ultrasound spectral image.

3. The method of claim 2, wherein the one or more evaluation parameters including cardiac function evaluation parameters related to systole and cardiac function evaluation parameters related to diastole, and the determining the one or more evaluation parameters for evaluating a function of the heart based on the plurality of cardiac cycles and the Doppler ultrasound spectral image includes:
determining the cardiac function evaluation parameters related to the systole based on the plurality of cardiac cycles and the first Doppler ultrasound spectral image; and
determining the cardiac function evaluation parameters related to the diastole based on the cardiac function evaluation parameters related to the systole and the second Doppler ultrasound spectral image.

4. The method of claim 3, wherein the determining the one or more evaluation parameters for evaluating a function of the heart based on the plurality of cardiac cycles and the Doppler ultrasound spectral image includes:
determining first target key point positions of the systole in each cardiac cycle of the plurality of cardiac cycles of the first Doppler ultrasound spectral image using a key point detection model, wherein the key point detection model is a machine learning model, and the first target key point positions are points for extracting the cardiac function evaluation parameters related to the systole;
determining second target key point positions in the diastole in each cardiac cycle of the plurality of cardiac cycles of the second Doppler ultrasound spectral image based on the cardiac function evaluation parameters related to the systole using the key point detection model, wherein the second target key point positions are points for extracting cardiac function evaluation parameters related to the diastole; and
determining the one or more evaluation parameters based on the first target key point positions and the second target key point positions.

5. The method of claim 1, wherein the determining a plurality of cardiac cycles based on the Doppler ultrasound spectral image includes:
performing, based on a horizontal axis in the Doppler ultrasound spectral image, a segmentation on the Doppler ultrasound spectral image to obtain a first Doppler ultrasound spectral image above the horizontal axis; and
determining the plurality of cardiac cycles based on the first Doppler ultrasound spectral image.

6. The method of claim 5, wherein the determining the plurality of cardiac cycles based on the first Doppler ultrasound spectral image includes:
generating an intermediate curve based on the first Doppler ultrasound spectral image;
determining a plurality of minimum value positions in the intermediate curve; and
determining the plurality of cardiac cycles based on the plurality of minimum value positions.

7. The method of claim 6, wherein the determining a plurality of minimum value positions in an intermediate curve generated based on the first Doppler ultrasound spectral image includes:
binarizing the first Doppler ultrasound spectral image;
summing pixel values on a vertical axis of the binarized first Doppler ultrasound spectral image and performing smoothing processing to obtain the intermediate curve; and
determining the plurality of minimum value positions based on the intermediate curve.

8. The method of claim 5, wherein the determining the one or more evaluation parameters for evaluating a function of the heart based on the plurality of cardiac cycles and the Doppler ultrasound spectral image includes:
dividing the first Doppler ultrasound spectral image into a plurality of first sub-images based on the plurality of cardiac cycles;
for each first sub-image of the plurality of first sub-images, determining a first key point heatmap set of the first sub-image and a first score of the first sub-image by inputting the first sub-image into a key point detection model, wherein the key point detection model is a machine learning model, and the first score reflects a recognition quality of a target key point in the first sub-image, the target key point is a point for extracting cardiac function evaluation parameters related to a cardiac cycle;
determining one or more first target sub-images from the plurality of first sub-images based on first scores of the plurality of first sub-images; and
determining first target key point positions of systole in a cardiac cycle corresponding to each first target sub-image of the one or more first target sub-images by post-processing the first key point heatmap set of the first target sub-image, the first target key point positions are points for extracting cardiac function evaluation parameters related to the systole.

9. The method of claim 8, wherein the first target key point positions of the systole includes a peak position of systolic motion velocity, a start position of an ejection phase, and an end position of the ejection phase, wherein the determining the one or more evaluation parameters for evaluating a function of the heart based on the plurality of cardiac cycles and the Doppler ultrasound spectral image further includes:
mapping peak positions of systolic motion velocity, start positions of the ejection phase, and end positions of the ejection phase in a plurality of first cardiac cycles to the Doppler ultrasound spectral image, wherein each of the plurality of first cardiac cycles is a cardiac cycle corresponding to each of the one or more first target sub-images;
determining a plurality of second sub-images by performing a period division on a second Doppler ultrasound spectral image that is below the horizontal axis in the Doppler ultrasound spectral image based on the peak positions of systolic motion velocity corresponding to the plurality of first cardiac cycles;
for each second sub-image of the plurality of second sub-images, determining a second key point heatmap set of the second sub-image and a second score of the second sub-image by inputting the second sub-image into the key point detection model, the second score reflects a recognition quality of the target key point in the second sub-image;
determining one or more second target sub-images from the plurality of second sub-images based on second scores of the plurality of second sub-images; and
determining second target key point positions in diastole in a cardiac cycle corresponding to each second target sub-image of the one or more second target sub-images by post-processing the second key point heatmap set of the second target sub-image, the second target key point positions are points for extracting cardiac function evaluation parameters related to the diastole.

10. The method of claim 9, wherein the second target key point positions in the diastole includes a peak position and a start position of early diastolic motion velocity, and a peak position and an end position of late diastolic motion velocity, wherein the determining the one or more evaluation parameters for evaluating a function of the heart based on the plurality of cardiac cycles and the Doppler ultrasound spectral image further includes:
determining a target Doppler ultrasound spectral image by marking the peak positions of systolic motion velocity, the start positions and the end positions of the ejection phase, peak positions and start positions of early diastolic motion velocity, and peak positions and end positions of late diastolic motion velocity in a plurality of second cardiac cycles on the Doppler ultrasound spectral image, wherein each of the plurality of second cardiac cycles is a cardiac cycle corresponding to each of the one or more second target sub-images; and
determining the one or more evaluation parameters based on the target Doppler ultrasound spectral image.

11. The method of claim 10, wherein the one or more evaluation parameters includes at least one of a peak systolic tissue velocity S', a peak early diastolic tissue velocity e', a peak late diastolic tissue velocity a', an isovolumic contraction time (IVCT), an ejection time (ET), and an isovolumic relaxation time (IVRT), wherein
determining the one or more evaluation parameters based on the target Doppler ultrasound spectral image includes at least one of:
determining a velocity corresponding to a peak position of systolic motion velocity in the target Doppler ultrasound spectral image as the peak systolic tissue velocity S';
determining a velocity corresponding to a peak position of early diastolic motion velocity in the target Doppler ultrasound spectral image as the peak early diastolic tissue velocity e';
determining a velocity corresponding to a peak position of late diastolic motion velocity in the target Doppler ultrasound spectral image as the peak late diastolic tissue velocity a';
determining a time from an end position of late diastolic motion velocity to a start position of an ejection phase adjacent on a right side in the target Doppler ultrasound spectral image as the IVCT;
determining a time between the start position and the end position of the ejection phase in the target Doppler ultrasound spectral image as the ejection time; or
determining a time from the end position of the ejection phase to a start position of early diastolic motion velocity of an early diastole adjacent on a right side in the target Doppler ultrasound spectral image as the IVRT.

12. The method of claim 11, further comprising:
determining a target peak systolic tissue velocity S'’ by calculating an average value of peak systolic tissue velocities S' in the plurality of cardiac cycles, or by determining a peak systolic tissue velocity S' in one cardiac cycle based on a first selection instruction;
determining a target peak early diastolic tissue velocity e'’ by calculating an average value of peak early diastolic tissue velocities e' in the plurality of cardiac cycles, or by determining a peak early diastolic tissue velocity e' in one cardiac cycle based on a second selection instruction;
determining a target peak late diastolic tissue velocity a'’ by calculating an average value of peak late diastolic tissue velocities a' in the plurality of cardiac cycles, or by determining a peak late diastolic tissue velocity a' in one cardiac cycle based on a third selection instruction;
determining a target IVCT by calculating an average value of IVCTs in the plurality of cardiac cycles, or by determining an IVCT in one cardiac cycle based on a fourth selection instruction;
determining a target ejection time by calculating an average value of ejection times in the plurality of cardiac cycles, or by determining an ejection time in one cardiac cycle based on a fifth selection instruction; and
determining a target IVRT by calculating an average value of IVRTs in the plurality of cardiac cycles, or by determining an IVRT in one cardiac cycle based on a sixth selection instruction.

13. The method of claim 10, further comprising:
calculating a standard deviation of target parameters in the plurality of second cardiac cycles, wherein the target parameters are at least one of the one or more evaluation parameters; and
determining a plurality of target cardiac cycles from the plurality of second cardiac cycles based on the standard deviation of the target parameters.

14. The method of claim 8, wherein training samples of the key point detection model includes a first training sample set, a second training sample set, a third training sample set, and a fourth training sample set, wherein
the first training sample set includes a plurality of sample first sub-images, the second training sample set includes a plurality of sample second sub-images, the third training sample set includes a plurality of complete sample first sub-images and a plurality of incomplete sample first sub-images, and the fourth training sample set includes a plurality of complete sample second sub-images and a plurality of incomplete sample second sub-images, and a training process of the key point detection model includes:
performing a plurality of iterations on an initial key point detection model based on a target loss function using the first training sample set, the second training sample set, the third training sample set, and the fourth training sample set until a convergence condition is reached, wherein
the target loss function includes a first loss term, a second loss term, a third loss term, and a fourth loss term, the first loss term reflecting a positioning accuracy of a target key point position in the systole, the second loss term reflecting a positioning accuracy of a target key point position in a diastole, the third loss term reflecting an accuracy of quality assessment of each sample first sub-image, and the fourth loss term reflecting an accuracy of quality assessment of each sample second sub-image.

15. The method of claim 14, wherein the target loss function further includes a fifth loss term, the fifth loss term reflecting a local consistency and a unimodality of a predicted key point.

16. The method of claim 14, wherein the training process of the key point detection model further includes:
determining a convergence speed of each loss term during one or more preceding rounds of training;
determining a weight corresponding to each loss term based on the convergence speed of each loss term; and
determining a value of the target loss function by weighting and summing all loss terms of the target loss function based on weights of the all loss terms.

17. A system, comprising:
at least one storage device storing executable instructions for determining one or more evaluation parameters of a heart, and
at least one processor in communication with the at least one storage device, when executing the executable instructions, causing the system to perform operations including:
obtaining a Doppler ultrasound spectral image of the heart;
determining a plurality of cardiac cycles based on the Doppler ultrasound spectral image; and
determining the one or more evaluation parameters for evaluating a function of the heart based on the plurality of cardiac cycles and the Doppler ultrasound spectral image.

18. The system of claim 17, wherein the determining a plurality of cardiac cycles based on the Doppler ultrasound spectral image includes:
dividing, based on a horizontal axis in the Doppler ultrasound spectral image, the Doppler ultrasound spectral image into a first Doppler ultrasound spectral image and a second Doppler ultrasound spectral image, the first Doppler ultrasound spectral image being a portion of the Doppler ultrasound spectral image above the horizontal axis, the second Doppler ultrasound spectral image being a portion of the Doppler ultrasound spectral image below the horizontal axis; and
determining the plurality of cardiac cycles based on the first Doppler ultrasound spectral image and the second Doppler ultrasound spectral image.

19. The method of claim 18, wherein the one or more evaluation parameters including cardiac function evaluation parameters related to systole and cardiac function evaluation parameters related to diastole, and the determining the one or more evaluation parameters for evaluating a function of the heart based on the plurality of cardiac cycles and the Doppler ultrasound spectral image includes:
determining the cardiac function evaluation parameters related to the systole based on the plurality of cardiac cycles and the first Doppler ultrasound spectral image; and
determining the cardiac function evaluation parameters related to the diastole based on the cardiac function evaluation parameters related to the systole and the second Doppler ultrasound spectral image.

20. A non-transitory computer readable medium, comprising at least one set of instructions for determining one or more evaluation parameters of a heart, wherein when executed by at least one processor of a computing device, the at least one set of instructions direct the at least one processor to perform operations including:
obtaining a Doppler ultrasound spectral image of the heart;
determining a plurality of cardiac cycles based on the Doppler ultrasound spectral image; and
determining the one or more evaluation parameters for evaluating a function of the heart based on the plurality of cardiac cycles and the Doppler ultrasound spectral image.
